(19) United States
(12) Patent Application Publication
(10) Pub. No.: US 2022/0257583 A1
(43) Pub. Date: Aug. 18, 2022

(54) Compositions and Methods for the Treatment of Intracranial Diseases

(71) Applicant: Duke University, Durham, NC (US)

(72) Inventors: Peter Fecci, Durham, NC (US); Pakawat Chongsathidkiet, Durham, NC (US); Alem Kahsai, Durham, NC (US); Robert Lefkowitz, Durham, NC (US)

(21) Appl. No.: 17/630,659

(22) PCT Filed: Jul. 31, 2020
(86) PCT No.: PCT/US20/44474

§ 371 Date: Jan. 27, 2022

Related U.S. Application Data
(60) Provisional application No. 62/881,468, filed on Aug. 1, 2019.
(60) Provisional application No. 62/885,514, filed on Aug. 12, 2019.

(51) Int. Cl. A61K 31/472; A61K 38/19; A61K 31/41; A61P 35/00; A61K 31/365; A61K 31/155; A61K 31/122; A61K 31/352; A61K 31/4045; A61K 31/423; A61K 31/196; A61K 31/12; A61K 31/343; A61K 31/44; A61K 31/58; A61K 31/4155

(52) U.S. Cl. CPC A61K 31/472; A61K 31/4155; A61K 31/41; A61P 35/00; A61K 31/365; A61K 31/155; A61K 31/122; A61K 31/352; A61K 31/4045; A61K 31/423; A61K 31/196; A61K 31/12; A61K 31/343; A61K 31/44; A61K 31/58; A61K 38/193

(57) ABSTRACT

Provided herein are compositions and methods for enhancing egress of T-cells from bone marrow of a subject in need thereof. Also provided are compositions and methods for the treatment of diseases characterized by reduced surface display of sphingosine-1-phosphate receptor 1 (S1P1), as well as methods of diagnosis/prognosis related to surface display of SIP 1. Methods of treating cancer are also provided.

### CROSS-RELATION TO RELATED APPLICATIONS

This application claims benefit of U.S. Provisional Patent Application No. 62/881,486, filed Aug. 1, 2019, and U.S. Provisional Patent Application No. 62/885,514, filed Aug. 12, 2019, and the contents of both application are herein incorporated in their entirety by reference.

### STATEMENT AS TO FEDERALLY SPONSORED RESEARCH

This invention was made with government support under Grant No. R01NS099096 awarded by the National Institutes of Health. The government has certain rights in the invention.

### TECHNICAL FIELD

The present invention generally relates to the technical fields of tumor biology, oncology, immunology, and medicine.

### BACKGROUND

A functional T-cell repertoire is a component of the initiation and maintenance of productive immune responses, e.g., anti-tumor immune responses. Disruptions to T-cell function (e.g., T-cell dysfunction) contribute to tumor immune escape, and to failure of the anti-tumor immune response in cancer patients. T-cell dysfunction is particularly severe in certain types of cancers such as glioblastoma (GBM), which is a common and potentially lethal primary malignant brain tumor. Despite near universal confinement to the intracranial compartment, GBM frequently depletes both the number and function of systemic T-cells. While severe T-cell lymphopenia (i.e., a decrease in the number of circulating T-cells) is a prominent characteristic of GBM, the cause of the lymphopenia is often attributed to treatment. Moreover, a lack of understanding of the mechanisms underlying T-cell dysfunction poses challenges to developing appropriate and meaningful therapeutic platforms.

Currently available treatments, including immunotherapies, for GBM and other intracranial diseases (e.g., tumors that have spread to the brain) have proven ineffective in part because of underlying T-cell dysfunction. Thus, there is an unmet need for therapies that effectively address the T-cell dysfunction component of such conditions.

### SUMMARY

The present invention relates to methods and compositions that can be useful in the treatment cancer.

Accordingly, in one aspect, the invention relates to a method of treating cancer, in a subject in need thereof, comprising interfering with activity of β-arrestin. In some embodiments, the method involves specifically interfering with the activity of β-arrestin2. IN some embodiments, the method involves administering an agent that inhibits β-arrestin2. In some embodiments, the agent is 1-(2-(6,7-dimethoxyisoquinolin-1-yl)methyl)-4,5-dimethoxyphenyl)ethan-1-one) (compound B29, also referred to herein as C29 or Cmpd29)) of general formula II:

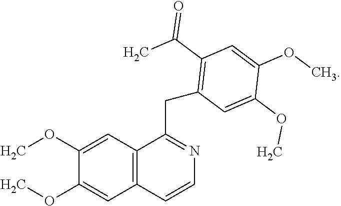

In another aspect, the invention relates to a method for treating an intracranial disease comprising enhancing egress of T-cells from bone marrow of a subject in need thereof. In some embodiments, the T-cells comprise surface displayed sphingosine-1-phosphate receptor 1 (S1P1), and wherein the method comprises increasing the interactions between S1P1 and sphingosine-1-phosphate (S1P). In some embodiments, the method comprises promoting S1P1 display on the surface of the T-cells. In some embodiments, the method comprises stabilizing S1P1 on the surface of the T-cells. In some embodiments, the method comprises reducing internalization of S1P1 from the surface of the T-cells. In some embodiments, the T-cells are naïve T-cells. In some embodiments, the T-cells are CD4 and/or CD8 T-cells. In some embodiments, the method comprises inhibiting an interaction between S1P1 and β-arrestin.

In some embodiments, the method comprises administering a β-arrestin inhibitor to the subject. In some embodiments, the β-arrestin inhibitor comprises a β-arrestin 1 inhibitor or a β-arrestin 2 inhibitor.

In some embodiments, the method comprises inhibiting GRK2-mediated phosphorylation of S1P1.

In some embodiments, the method comprises inhibiting clathrin-mediated endocytosis of S1P1.

In some embodiments, the method further comprises administering a 41BB agonist and/or a PD-1 blockade to the subject.

In some embodiments, the method further comprises administering a granulocyte colony-stimulating factor to the subject.

In some embodiments, the subject is a human.

In some embodiments, the intracranial disease is a primary intracranial tumor, an intracranial metastatic tumor, an inflammatory brain disease or disorder, a stroke, or a traumatic brain injury. In some embodiments, the intracranial disease is glioblastoma.

In another aspect, the invention relates to a pharmaceutical composition comprising an agent that promotes surface display of sphingosine-1-phosphate receptor 1 (S1P1) on a T-cell. In some embodiments, the agent increases the interaction between S1P1 and sphingosine-1-phosphate (S1P). In some embodiments, the agent stabilizes S1P1 on the surface of the T-cell. In some embodiments, the agent reduces internalization of S1P1 from the surface of the T-cell. In some embodiments, the agent inhibits an interaction between S1P1 and arrestin.

In some embodiments, the agent comprises a β-arrestin inhibitor. In some embodiments, the agent comprises a β-arrestin 1 inhibitor or a β-arrestin 2 inhibitor. In some embodiments, the agent inhibits GRK2-mediated phosphorylation of S1P1. In some embodiments, the agent inhibits clathrin-mediated endocytosis of S1P1. In some embodiments, the agent is (Z)-3-((furan-2-ylmethyl)imino)-N,N-dimethyl-3H-1,2,4-dithiazol-5-amine) (compound C30) of general formula I:

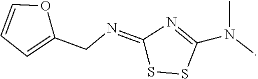

In some embodiments, the agent is a β-arrestin 2 inhibitor. In some embodiments, the agent is 1-(2-((6,7-dimethoxyisoquinolin-1-yl)methyl)-4,5-dimethoxyphenyl)ethan-1-one) (compound B29, also referred to herein as C29 or Cmpd29)) of general formula II:

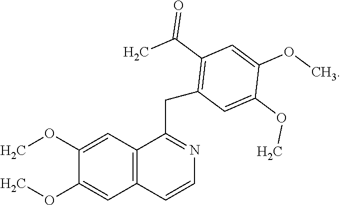

In some embodiments, the inhibitor is any one of the compounds shown by general formula in FIG. 23 and recited by IUPAC name in Table 3 herein, or any combination thereof.

In another aspect, the invention relates to a method of treating a disease or a disorder associated with T-cell sequestration in the bone marrow in a subject in need thereof, the method comprising administering a pharmaceutical composition comprising a β-arrestin inhibitor in an amount effective to release the T-cells from sequestration.

In another aspect, the invention relates to a method of treating a disease or a disorder associated with loss of sphingosine-1-phosphate receptor 1 (S1P1) expression on the surface of T-cells in a subject in need thereof, the method comprising administering a β-arrestin inhibitor in an amount effective to stabilize S1P1 levels on the T-cells by hindering S1P1 internalization.

In another aspect, the invention relates to a method for mobilizing T-cells sequestered in the bone marrow into circulation in a subject in need thereof, the method comprising administering a β-arrestin inhibitor in an amount effective to release the T-cells into circulation.

In another aspect, the invention relates to a method for reversing T-cell ignorance in a subject in need thereof, the method comprising administering a β-arrestin inhibitor in an amount effective to stabilize S1P1 levels on the T-cells, thereby reversing the ignorance.

In another aspect, the invention relates to a method for treating cancer in a subject in need thereof, comprising administering a β-arrestin inhibitor.

In another aspect, the invention relates to a method of diagnosis of intracranial tumors, the method comprising determining the presence of S1P1 on the surface of T cells, wherein a loss of surface S1P1 on the T cells indicates the presence of or advancement of the intracranial tumor.

### BRIEF DESCRIPTION OF THE DRAWINGS

FIGS. 1A-1B: T-cell lymphopenia and splenic contraction in treatment-naïve subjects with GBM. FIG. 1A: Blood CD4⁺ and CD8⁺ T-cell counts measured prospectively in n=15 newly diagnosed subjects with GBM (before therapy) and n=13 age-matched controls. FIG. 1B: Spleen volume on abdominal computed tomography scans performed on n=278 newly diagnosed treatment-naïve subjects with GBM and n=43 age-matched controls. All data in FIG. 1A and FIG. 1B are shown as mean±s.e.m. P values were determined by two-tailed, unpaired Student's t-test.

FIGS. 1C-1F: FIG. 1C: Frequency of lymphopenia (lymphocyte counts <1,000 cells/μL) in n=300 newly diagnosed GBM patients and n=46 age-matched controls. GBM patients are also categorized into n=97 Dexamethasone-experienced (Dex) and n=187 Dexamethasone-naïve (No Dex) groups. FIG. 1D: Comparison of naïve (CD45RA⁺CD27⁺) and memory (CD45RA⁻) CD4⁺ T-cell counts in n=13 GBM patients versus n=11 controls. Both naïve and memory CD4⁺ T-cell counts are reduced in patients, with the naïve T-cell loss being proportionately more severe. FIG. 1E: Ratio of naïve to memory CD4⁺ T-cell counts in the same cohorts of n=13 GBM patients versus n=11 controls. Disproportionate naïve T-cell loss resulted in trend towards lower ratios in patients. FIG. 1F: Spleen volume on CT scans in n=176 dexamethasone-naïve (No Dex) and n=91 dexamethasone-experienced (Dex) GBM patients. Data shown as mean±s.e.m. P values were determined by two-tailed, Fisher's exact test (FIG. 1C), two-tailed, unpaired Student's t-test (FIG. 1D, FIG. 1F), and two-tailed, Mann Whitney test with Gaussian approximation (FIG. 1E).

FIGS. 2A-2D: Recapitulated T-cell lymphopenia and lymphoid organ contraction in murine glioma. FIG. 2A: Blood CD4 T-cell counts in n=8 control C57BL/6 and n=5 control VM/Dk mice, or n=9 intracranial CT2A glioma-bearing C57BL/6 mice and n=10 SMA-560 glioma-bearing VM/Dk mice. FIG. 2B: Blood CD8 T-cell counts in n=8 control C57BL/6 and n=5 control VM/Dk mice, or n=9 intracranial CT2A glioma-bearing C57BL/6 mice and n=9 SMA-560 glioma-bearing VM/Dk mice. Data in FIG. 2A and FIG. 2B are shown as mean±s.e.m. P values determined by two-tailed, unpaired Student's t-test. FIG. 2C: Gross image depicting spleens taken from unimplanted or intracranial CT2A glioma-bearing C57BL/6 mice. FIG. 2D: H&E staining (upper panel) or immuno-histochemistry for CD3 (lower panel) of formalin-fixed paraffin-embedded spleen taken from unimplanted or intracranial CT2A glioma-bearing C57BL/6 mice. Histopathologic examination of spleens from intracranial CT2A mice showed diminution in T-cell-dependent lymphoid areas. These findings accompanied marked organ lymphopenia and lymphoid necrosis. Immunohistochemistry confirmed that spleens of intracranial CT2A mice had marked T-cell lymphopenia. The scale bar is 200 μm. All data in FIG. 2A-2D are representative findings from one of at least three independently repeated experiments with similar results. Blood (FIG. 2A, FIG. 2B) was drawn and spleens (FIG. 2C, FIG. 2D) were harvested at 18 d following tumor implantation (IC=intracranial.)

FIGS. 2E-2H: FIG. 2E: Blood naïve (CD44⁻CD62L⁺) and memory (CD44⁺) CD4⁺ T-cell counts depicted for n=7 control C57BL/6 and n=9 IC CT2A IC mice. FIG. 2AF: Spleen T-cell counts in n=10 control C57BL/6 and n=6 control VM/Dk mice or n=14 IC CT2A glioma-bearing C57BL/6 and n=8 IC SMA-560 glioma-bearing VM/Dk mice. Data are shown as mean±s.e.m. P values and were determined by two-tailed, unpaired Student's t-test. FIG. 2G: Gross image depicting thymuses taken from unimplanted or IC CT2A glioma-bearing C57BL/6 mice. FIG. 2H: H&E staining (upper panel) or IHC for CD3 (lower panel) of FFPE thymus taken from unimplanted or IC CT2A glioma-bearing C57BL/6 mice. Histopathologic examination of thymus from IC CT2A mice showed loss of normal cortico-medullary architecture. These findings accompanied marked organ lymphopenia and lymphoid necrosis. IHC confirmed thymus of IC CT2A mice has marked T-cell lymphopenia, for H&E, scale bar=50 μm; for IHC, scale bar=200 All data in E-H are representative findings from one of at least three independently repeated experiments with similar results. Both blood draw (FIG. 2E) and spleen/thymus harvest (FIG. 2F-FIG. 2H) were performed at 18 days following tumor implantation.

FIGS. 3A-3H: Naïve T-cells accumulate in the bone marrow of mice and subjects with GBM. FIG. 3A: Bone marrow T-cell counts from a single hind leg femur and tibia in n=4 control C57BL/6 and n=8 control VM/Dk mice, or n=13 intracranial CT2A glioma-bearing C57BL/6 mice and n=14 SMA-560 glioma-bearing VM/Dk mice. FIG. 3B: Bone marrow CD4+ and CD8+ T-cell counts in n=4 control C57BL/6 or n=13 intracranial CT2A mice. FIG. 3C: Bone marrow naïve and memory CD4+ T-cell counts in n=3 control C57BL/6 or n=13 intracranial CT2A mice. Cumulative data from three experiments are depicted in FIG. 3A-3C. FIG. 3D: The ratios of bone marrow to blood CD4 and CD8 counts were calculated for n=15 treatment-naïve GBM subjects and n=13 spinal fusion controls.

FIG. 3E: For the same n=13 controls and n=15 GBM subjects, paired absolute CD4+ T-cell counts in blood and bone marrow are depicted. Median counts in each compartment are identified by horizontal lines. Dashed line demarcates low cut-off of normal CD4 range. Similar results were obtained for CD8+ T-cells. FIG. 3AF: For the same n=13 controls and n=15 GBM subjects, the ratio of bone marrow to blood naïve and memory T-cells was calculated. FIG. 3G: Treg cell counts in the bone marrow of n=11 controls compared to n=15 GBM subjects. FIG. 3H: Relative frequencies of CD4+ helper T-cell subsets: Th1 (CXCR3+CCR6−), Th2 (CXCR3−CCR6−), and Th17 (CXCR3−CCR6+) in bone marrow of n=13 controls and n=15 GBM subjects. Data in FIG. 3A-3D and FIG. 3F-3H are shown as mean±s.e.m. P values were determined by two-tailed, unpaired Student's t-test (FIG. 3A-3C, 3G, 3H) and two-tailed Mann-Whitney test with Gaussian approximation (FIG. 3D, 3F). Blood and bone marrow CD4+ T-cell counts in FIG. 3E were compared using Wilcoxon matched-pairs signed rank tests. P values are depicted. BM, bone marrow.

FIGS. 3I-3L: FIG. 3I: Sample flow cytometry plot examining bone marrow T-cells in control C57BL/6 mice (top), or the same mice bearing IC CT2A (bottom). FIG. 3J: Frequency of additional leukocyte populations in the bone marrow of n=5 control C57BL/6 or n=3 IC CT2A glioma-bearing C57BL/6 mice. Data in FIG. 3I are representative findings from one of at least three independently repeated experiments with similar results. Data in bottom three boxes are cumulative results from two experiments. Data in FIG. 3J are shown as mean±s.e.m. P values were determined by two-tailed, unpaired Student's t-test. FIG. 3K: Time course of T-cell accumulation in the bone marrow of mice bearing IC CT2A after tumor implantation. Bone marrow were harvest from n=3 IC CT2A glioma mice on day 0, 9, 15 and n=4 IC CT2A glioma mice on day 21 after tumor implantation. Data in FIG. 3K are representative findings from one of at least three independently repeated experiments with similar results. Data shown as mean±s.e.m. P values were determined by two-tailed, unpaired Student's t-test. FIG. 3L: Sample flow cytometry plot examining bone marrow T-cells. The relative proportions of central memory (CM), naïve (N), effector memory (EM), and terminal effector (TE) populations in a patient blood (top) and bone marrow (bottom). CD4⁺ T-cells are depicted. Similar results were obtained for CD8⁺ T-cells.

FIGS. 4A-4F: T-cell accumulation in bone marrow reflects intracranial tumor location rather than tumor histologic type. FIG. 4A: Bone marrow T-cell counts in n=13 subcutaneous and n=17 intracranial CT2A glioma-bearing C57BL/6 mice, or n=15 subcutaneous and n=13 intracranial E0771 breast carcinoma-bearing mice, or n=9 subcutaneous and n=9 intracranial B16F10 melanoma-bearing mice, or n=13 subcutaneous and n=12 intracranial LLC-bearing mice. FIG. 4B: CD8/CD4 ratios in the bone marrow of n=15 intracranial CT2A, n=9 intracranial E0771, n=9 intracranial B16F10, or n=12 intracranial LLC-bearing mice. FIG. 4C: Naïve/memory T-cell ratios in the bone marrow of the same tumor-bearing mice as in b. Counts and ratios in FIG. 4A-FIG. 4C were compared to those in the bone marrow of n=17 control C57BL/6 mice. Data in FIG. 4A-FIG. 4C are cumulative results from a minimum of two experiments with each tumor type. FIG. 4D: Accumulation of adoptively transferred CFSE-labeled T-cells in the bone marrow of n=5 recipient control C57BL/6 or CT2A glioma-bearing C57BL/6 mice. Glioma-bearing mice harbored tumors in either the intracranial or subcutaneous compartment (n=3 tumor-bearing mice per group). T-cell counts were assessed 24 h following adoptive transfer. Transferred cells were splenocytes from naïve C57BL/6 (control) donors. FIG. 4E: Accumulation of adoptively transferred CFSE-labeled T-cells in the bone marrow of n=5 control recipient mice and n=8 intracranial CT2A-bearing (CT2A IC) recipient mice at 2 h (left) post-transfer, or n=7 control recipient mice and n=14 intracranial CT2A-bearing (CT2A IC) recipient mice at 24 h (right) post-transfer. Transferred cells were splenocytes from naïve C57BL/6 (control) donors. FIG. 4F: Accumulation of adoptively transferred CFSE-labeled T-cells in the bone marrow of n=5 control recipient mice and n=6 CT2A IC recipient mice 24 h after transfer (left). Transferred cells were splenocytes from naïve C57BL/6 (control) donors. Accumulation of adoptively transferred CFSE-labeled T-cells in the bone marrow of n=3 control recipient mice and n=5 CT2A IC recipient mice 24 h after transfer (right). Transferred cells were bone marrow cells from CT2A IC mice. Data in FIG. 4A-4F are representative findings from one of a minimum of two independently repeated experiments with similar results. Data in FIG. 4A-4F are shown as mean+s.e.m. P values in FIG. 4A and FIG. 4D-4F were determined by two-tailed, unpaired Student's t-test. Ratios in FIG. 4B and FIG. 4C were compared using one-way ANOVA, with post hoc Tukey's test when applicable. P values are depicted. SC, subcutaneous.

FIGS. 4G-4H: FIG. 4G: Bone marrow T-cell counts were compared among n=5 control un-operated C57BL/6 mice and n=5 C57BL/6 mice receiving sham IC injections of a saline/methylcellulose mixture. No difference in bone marrow T-cell counts was observed. Data depicted are from Day 18 post-injection. Data in FIG. 4G are representative findings from one of two independently repeated experiments with similar results. Data are shown as mean±s.e.m. P values were determined by two-tailed, unpaired Student's t-test. FIG. 4H: Pictorial schematic for the experiments producing the data depicted in FIG. 4A-4F.

FIGS. 5A-5G: Loss of surface S1P1 on T-cells directs their sequestration in bone marrow in the setting of intracranial tumors. FIG. 5A: The percentage of nascent T-cells expressing surface S1P1 was assessed by flow cytometry in the bone marrow of n=6 control C57BL/6 mice or n=6 mice bearing intracranial CT2A on day 18 following tumor implantation. FIG. 5B: Representative flow cytometry plot of data depicted in FIG. 5A and FIG. 5C, Negative correlation between bone marrow T-cell counts and S1P1 levels on bone marrow T-cells across intracranial and subcutaneous murine tumor models. Data in FIG. 5C were obtained from n=6 intracranial CT2A, n=5 intracranial E0771, n=6 intracranial B16F10, n=7 intracranial LLC, n=6 subcutaneous CT2A, n=7 subcutaneous E0771, n=6 subcutaneous B16F10, and n=7 subcutaneous LLC tumor-bearing mice. N=21 control C57BL/6 mice were also included. Data in FIG. 5C are cumulative results from a minimum of two experiments with each tumor type. FIG. 5D: The percentage of nascent T-cells expressing surface S1P1 was assessed by flow cytometry in the bone marrow of n=14 GBM subjects or n=12 age-matched controls. FIG. 5E: Representative flow cytometry plot of data depicted in FIG. 5D and FIG. 5F, Negative correlation between bone marrow T-cell counts and surface S1P1 levels on bone marrow T-cells in n=12 GBM subjects and n=10 age-matched controls. FIG. 5G: Relative sequestration of adoptively transferred CFSE-labeled T-cells within the bone marrow of intracranial CT2A recipient mice either 2 h or 24 h after transfer (n=5 mice per group). As indicated, transferred cells were splenocytes either from control C57BL/6 donors (control) or from S1P1 conditional knockout (S1P1-KO) donors. Data in FIG. 5G are representative findings from one of a minimum of two independently repeated experiments with similar results. All data in FIG. 5A, FIG. 5D, and FIG. 5G are shown as mean±s.e.m. P values and were determined by two-tailed, unpaired Student's t-test. Two-tailed P values and Pearson coefficients for FIG. 5C and FIG. 5F are depicted.

FIGS. 5H-5P: FIG. 5H: Bone marrow T-cell counts are shown for n=9 control tumor-naïve C57BL/6 and n=13 IC CT2A-bearing mice, or n=5 control tumor-naïve C57BL/6 and n=8 IC CT2A-bearing mice administered the CXCR4 antagonist AMD3100 (AMD). FIG. 5I: Frequency of S1P1⁺ T-cell populations in the spleen (left) and cervical lymph nodes (CLN) (right) of n=12 control C57BL/6 or n=6 CT2A IC mice. FIG. 5J: S1Pr1 mRNA expression levels in T-cells sorted from spleens of n=3 control C57BL/6 or n=3 CT2A IC mice assessed by qRT-PCR and normalized to GAPDH expression. FIG. 5K: Histograms showing expression levels of CD69, KLF2, and STAT3 in the T-cells of bone marrow of control C57BL/6 (gray) or CT2A IC (black) mice assessed by RNA prime flow. Data in FIG. 5H-FIG. 5K are representative findings from one of two independently repeated experiments with similar results. FIG. 5L: Concentration of S1P1 ligand in the plasma of n=5 control C57BL/6 or n=6 IC CT2A-bearing mice, as assessed by LC-MS/MS. FIG. 5M: Concentration of S1P1 ligand in the brain or brain tumor of n=5 control C57BL/6 or n=7 IC CT2A-bearing mice, as assessed by LC-MS/MS. Data in FIG. 5M are normalized to tissue weight. Data in FIG. 5H-FIG. 5J, FIG. 5L and FIG. 5M are shown as mean±s.e.m. All P values were determined by two-tailed, unpaired Student's t-test. FIG. 5N-FIG. 5O: Negative correlation between bone marrow T-cell counts and either spleen (FIG. 5N) or thymus (FIG. 5O) weight across IC and SC murine tumor models. Data in FIG. 5N were obtained from n=6 IC CT2A, n=9 IC E0771, n=6 IC B16F10, n=7 IC LLC, n=6 SC CT2A, n=10 SC E0771, n=11 SC B16F10, and n=7 SC LLC tumor-bearing mice. N=21 control C57BL/6 were also included. Data in FIG. 5O were obtained from n=6 IC CT2A, n=5 IC E0771, n=6 IC B16F10, n=7 IC LLC, n=6 SC CT2A, n=7 SC E0771, n=6 SC B16F10, and n=7 SC LLC tumor-bearing mice. N=21 control C57BL/6 were also included. Data in FIG. 5N and FIG. 5O are cumulative results from a minimum of two experiments with each tumor type. Two-tailed, p values and Pearson coefficients for FIG. 5N and FIG. 5O are depicted. FIG. 5P: Accumulation of adoptively transferred CFSE-labeled T-cells in the bone marrow of CT2A IC recipients that were treated with either vehicle control (n=3 recipient mice) or FTY720 (n=3 recipient mice) 2 hours prior to receiving transfers. Transferred cells were splenocytes from control C57BL/6 donors. Data in FIG. 5P are shown as mean±s.e.m. The p value was determined by two-tailed, unpaired Student's t-test.

FIGS. 6A-6E: Hindering S1P1 internalization abrogates T-cell sequestration in bone marrow. FIG. 6A: Relative sequestration of adoptively transferred CFSE-labeled T-cells within the bone marrow of CT2A IC recipient mice at 2 h (left) or 24 h (right) after transfer. As indicated, transferred cells were splenocytes either from control C57BL/6 donors (control) or from S1P1 stabilized knock-in (S1P1 KI) donors (n=5 recipient mice per group). Data in FIG. 6A are representative findings from one of a minimum of two independently repeated experiments with similar results. FIG. 6B: T-cell counts in the bone marrow of n=10 intracranial CT2A-bearing wild-type C57BL/6 or n=11 S1P1 KI mice. Counts were assessed at 18 d following tumor implantation and are shown relative to baseline counts inn=6 tumor-naïve control wild-type or n=6 tumor-naïve S1P1 KI mice. Cumulative data from three experiments are depicted in FIG. 6B and FIG. 6C, Intracranial CT2A tumors were harvested from n=6 wild-type C57BL/6 (WT) or n=6 S1P1 KI mice at 18 d following tumor implantation. TILs were assessed by flow cytometry and the number of total T-cells per gram of tumor quantified. FIG. 6D: The frequency of activated effector) (CD44^{hi}CD62L^{lo}) T-cells in intracranial CT2A tumors from the same n=6 wild-type C57BL/6 (WT) or n=6 S1P1 KI mice in FIG. 6C was also quantified. Data in FIG. 6C and FIG. 6D are representative findings from one of a minimum of three independently repeated experiments with similar results. FIG. 6E: C57BL/6 (WT) or S1P1 KI mice were implanted with intracranial CT2A tumors and treated with a 4-1BB agonist antibody or isotype control (n=8 per group). All data in FIG. 6A-FIG. 6D are shown as mean±s.e.m. P values in and were determined by two-tailed, unpaired Student's t-test. Survival in FIG. 6E was assessed by two-tailed, generalized Wilcoxon test. P value for overall comparison is depicted.

FIGS. 6F-6J: FIG. 6F: Representative flow cytometry plot depicting the frequency of S1P1 on the surface of T-cells in the bone marrow of C57BL/6 mice and S1P1 KI bearing IC CT2A tumor. FIG. 6G: S1P1 KI mice were implanted with IC CT2A tumors and treated with anti (α)-PD-1, 4-1BB agonist, or the combination of both (n=8 per group). FIG. 6H and FIG. 6I: Bone marrow (FIG. 6H) and blood (FIG. 6I) T-cell counts in n=8 control mice and n=8 IC CT2A-bearing mice administered control treatment or G-CSF intraperitoneally every 3 days following tumor implantation (Days 3-18). Counts were assessed 18 days following tumor implantation. FIG. 6J: IC CT2A IC mice were administered G-CSF, 4-1BB agonist, or the combination regimen of both drugs (n=8 per group). All data in FIG. 6F=FIG. 6J are representative findings from one of two independently repeated experiments with similar results. Data in FIG. 6H and FIG. 6I are shown as mean±s.e.m. P values and were determined by two-tailed, unpaired Student's t-test. Survivals in FIG. 6G and FIG. 6J were assessed by two-tailed generalized Wilcoxon test. P values for overall comparison are depicted.

FIG. 7: Adoptively transferred T-cells from both BARR1 and BARR2 knockout donors resist sequestration in bone marrow of CT2A glioma-bearing recipients. Naïve CFSE-labeled splenocytes (10′) from the indicated donors were adoptively transferred IV into IC CT2A-bearing recipient mice. The number of CFSE+ T-cells in the bone marrow of recipients was determined by flow cytometry 24 h later. While T-cells from control were sequestered, T-cells from S1P1-stabilized (KI) and β-arrestin 1 and 2 KO donors were not.

FIGS. 8A-8C: Bone marrow T-cell sequestration is abrogated in BARR2 knockout mice bearing murine CT2A glioma, but not BARR1 knockout mice FIG. 8A. Also exclusive to BARR2 knockout mice bearing CT2A was a restoration of T-cell S1P1 levels (FIG. 8B) and of spleen volumes (FIG. 8C) to nearly control levels.

FIG. 9: β-arrestin 2 knockout mice, but not β-arrestin 1 knockout mice, show improved survival in intracranial murine CT2A glioma model. FIG. 9 is a Kaplan-Meier survival curve of intracranial CT2A murine glioma model in wild type C57BL/6, βARR1 KO, and βARR2 KO mice (n=8 per group).

FIG. 10: Survival benefit of β-arrestin 2 knockout mice previously observed in intracranial murine CT2A glioma model is abrogated with CD8 T-cell depletion, suggesting β-arrestin 2 inhibition conveys survival benefits against intracranial tumors in a T-cell dependent manner.

FIGS. 11A and 11B: βARR2 depletion provides anti-tumor efficacies in various models of cancer. β-arrestin 2 knockout mice, but not β-arrestin 1 knockout mice, show restricted tumor growth in a subcutaneous murine CT2A glioma model, despite absence of T-cell sequestration in the context of subcutaneous tumors, indicating multiple benefits to β-arrestin 2 inhibition beyond just reversal of sequestration. FIG. 11A shows a plot of subcutaneous CT2A tumor volumes in wild type C57BL/6, βARR2 KO mice over time (n=3-4 per group). FIG. 11B shows a plot of subcutaneous B16F10 melanoma tumor volumes in wild type C57BL/6, βARR1 KO, and βARR2 KO mice over time (n=4-6 per group). B16F10 melanoma cells were grown and collected in the logarithmic growth phase. For subcutaneous implantation, 2.5×105 B16F10 cells were delivered in a total volume of 200 μl per mouse into the subcutaneous tissues of the left flank. The βARR2 KO cohort reveals delayed tumor growth.

FIG. 12: T cells exposed to higher concentrations of the non-specific β-arrestin small molecule antagonist “C30” (identified by the inventors through the screening process delineated in Example 10) demonstrated higher levels of S1P1 expression.

FIG. 13: The first 40 initial hits from the screening of a structurally diverse, drug-like compound (DDLC) library containing more than 3,500 unique molecules with binding activity against purified β-arrestin 2 were evaluated for β-arrestin 2 recruitment by DiscoveRx assay. DiscoveRx cells expressing chimeric β2-adrenergic receptor (β2AR) with C-terminal tail from vasopressin receptor 2 (β2V2R) that is known to bind β-arrestin 2 very tightly were employed in this assay. The DiscoveRx cells were pretreated with candidate compounds at 50 μM or DMSO (control) for 25 minutes followed by stimulation with isoproterenol (10 nM). Compound B29 inhibits more than 75% of β-arrestin 2 activity (red-dashed rectangle) induced by isoproterenol which is a receptor agonist (red bar graph).

FIG. 14: β-arrestin 2 recruitment to activated β2V2R. Testing B29 further, the DiscoveRx cells were pretreated with compound B29 at 1, 10, 50 μM or DMSO (control) for 25 minutes followed by stimulation with isoproterenol at various concentrations. The titration curves with β-arrestin 2 recruitment activity reveal that B29 shifts the potency of agonist rightward and decreases maximal response in a dose dependent fashion, indicating that it inhibits the β-arrestin 2-induced functional response.

FIGS. 15A and 15B: FIG. 15A shows Kaplan-Meier survival curves of intracranial E0771 triple negative breast cancer model in wild type C57BL/6 and βARR2 KO mice over time (n=9 per group). FIG. 15B shows a plot of subcutaneous E0771 tumor volumes in wild type C57BL/6, βARR1 KO, and βARR2 KO mice over time (n=4 per group).

FIG. 16 shows, together with FIG. 10, that βARR2 deficiency requires T-cells to convey a survival benefit in the setting of GBM. FIG. 16 shows that the survival benefit of βARR2 KO mice previously observed in intracranial murine CT2A glioma model is abrogated with CD4 T-cell depletion (FIG. 10 shows similar effect with CD8), suggesting that βARR2 inhibition conveys survival benefits against intracranial tumors in a T-cell dependent manner (n=8 per group).

FIGS. 17A and 17B show βARR2 depletion synergizes with 4-1BB agonism and checkpoint blockades. FIG. 17A shows Kaplan-Meier survival curves of intracranial CT2A murine glioma models in βARR2 KO mice treated with 4-1BB agonist antibody when compared to relevant controls (n=7-9 per group). FIG. 17B shows Kaplan-Meier survival curves of intracranial CT2A murine glioma models in βARR2 KO mice treated with an anti-PD1 antibody when compared to relevant controls (n=7-9 per group).

FIG. 18 is a schematic representation of the evaluation of small molecules against βARR1 and βARR2 using FSTA. Approximately 3,500 structurally diverse, drug-like compounds (DDLC) were screened against purified βarr1 or βarr2 at a compound concentration of 50 μM. The primary screen identified 80 hits that altered the thermal conformational stability of βarr1 or βarr2 by 2° C. compared to controls. Based on secondary confirmation binding, activity and toxicity assays, the 80 initial hits were reduced to 56 hits to undergo further characterization. 35 among which are common binders to both isoforms while 21 bind preferentially to βarr2 under such binding condition.

FIG. 19 is a graphic representation showing FSTA-based binding of 21 hits to βarrestin-1 or βarrestin-2. Plots of the change in melting thermal shift (ΔTm) of βarrs (βarr1 open bar graphs, βarr2 closed bar graphs) in presence of hit compounds (total 21 small-molecules that have preference to bind to barr2 over barr1 under this experimental setting). V2Rpp is a control; βarr1/2 binding phosphorylated peptide which corresponds to the C-terminus of the GPCR, vasopressin-2 receptor (V2R). Compounds scoring ΔTm values approximately ≥2 or ≤−2° C. were considered potential binders to βarr1/2 (dashed lines). All 21 bound preferentially to βarr2 isoform over βarr1.

FIG. 20 is a graphic representation showing the effect of putative βarr2 binding compounds (21 hits) on βarr2 recruitment to agonist activated GPCR. DiscoveRx-U2OS cells exogenously expressing βarr2 and β2V2R were treated with each putative βarrestin binding compound at 50 μM for 30 min and then stimulated with agonist isoproterenol (10 nM) to induce recruitment of βarr. Data are presented as means±SEM (n=5). The dashed line indicates control agonist alone induced response (10 nM). Above this line indicates compounds that enhance βarrs2 activities (activators) and below which compound that inhibit βarrs. Seventeen compounds inhibit isoproterenol-induced βarr2 recruitment to receptor. The remaining 4 either enhance βarr2 activities (C3, C58, and C78) or have little to no effect (C67).

FIG. 21 is a graphic representation showing effects of compounds on βarr2-promoted high-affinity agonist state of the GPCR, pβ2V2R. All 21 compounds were evaluated for their influence on βarr2-promoted high-affinity receptor state in radio-labeled agonist (³H-Fen) binding studies in vitro, using phosphorylated GPCR, β2V2R in membranes. Binding of an agonist at the orthosteric pocket of GPCRs has been previously shown to promote enhanced binding affinity of the βarrs as well as the bound agonist for the receptor. Exogenously added βarr2 enhanced the high-affinity agonist (³H-Fen) binding state of the pβ2V2R (second bar graph/open bar graph). Inhibitor decrease while activator (C3) increase this βarr-promoted high-affinity ³H-Fen binding signals (bar-graphs in black). The first bar graph in each panel is DMSO alone without βarr2. Dashed lines indicate control lines, above which indicates compound that activate and below which compound that inhibit βarr2. Boxed compounds didn't have inhibitory effects on βarr2-recruitment. All 17 inhibited βarr2-promoted high affinity agonist state of the receptor.

FIG. 22 is a graphic representation showing the effects of 17 βarr2-binders on βarr-dependent GPCR mediated ERK MAP kinase activation. Effect of 17 βarr2 binders on βarr-dependent, carvedilol-induced β2-adrenergic receptor (β2AR) mediated ERK phosphorylation in HEK293 cells stably expressing FLAG-tagged β₂ARs. Bar graphs showing quantification of ERK activation in presence of vehicle DMSO, 1 μM agonist isoproterenol (ISO), 10 μM of a βarr biased ligand Carvedilol (Cary), 30 μM the compounds alone or together with Carvedilol (Cary). HEK293 cells stably expressing FLAG-tagged β₂ARs were pretreated with vehicle or compounds for 30, then stimulated with indicated concentration of carvedilol for 5 min, quenched and analyzed by Western blotting. Data represent the mean±SEM for n independent experiments. DMSO no stimulation; Cary carvedilol; Iso isoproterenol; p-ERK phosphorylated ERK; t-ERK total ERK. Thirteen out of these 17 compounds inhibited Barr-dependent ERK activation while 4 have little to no effects. One compound among these was found to bind to receptor as well (C4) and C36 has cytotoxicity issues (it is an FDA approved drug).

FIG. 23 shows formulae for 15 compounds evaluated in FIG. 22, excluding C4 and C36 based on other criteria.

### DETAILED DESCRIPTION

Technical and scientific terms used herein have the meanings commonly understood by one of ordinary skill in the art to which the present invention pertains, unless otherwise defined. For example, The Concise Dictionary of Biomedicine and Molecular Biology, Juo, Pei-Show, 2nd ed. 2002, CRC Press; The Dictionary of Cell and Molecular Biology, 3rd ed., 1999, Academic Press; and The Oxford Dictionary of Biochemistry and Molecular Biology, Revised 2000, Oxford University Press, provide one of skill in the art with a general dictionary of many of the terms used herein. Additionally, commonly used molecular biology terms, methods and protocols are provided in Molecular Cloning: A laboratory manual, M. R. Green and J. Sambrook (eds.), 4th ed. 2012, Cold Spring Harbor Laboratory Press, New York. Additional definitions are set forth throughout the detailed description. Reference is made herein to various methodologies known to those of ordinary skill in the art. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted. Publications and other materials setting forth such known methodologies to which reference is made are incorporated herein by reference in their entireties as though set forth in full.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “subject” denotes any mammal, including humans.

As used herein, the phrase “effective amount” means an amount of composition that provides the specific effect for which the composition is administered. It is emphasized that an effective amount of the composition will not always be effective in ameliorating a disease, even though such amount is deemed to be an effective amount by those of skill in the art. Those skilled in the art can determine such amounts in accordance with standard practices as needed to treat a specific subject and/or condition/disease.

As described herein, the present disclosure relates to addressing the aforementioned challenges and unmet needs by providing, inter alia, compositions and methods for the treatment diseases characterized by reduced surface display of sphingosine-1-phosphate receptor 1 (S1P1). Exemplary diseases along these lines are intracranial diseases and other conditions (e.g., tumors, inflammation, stroke, traumatic brain injury) S1P1 surface display on T-cells.

### INTRODUCTION

It was surprisingly determined that treatment-naïve GBM patients and mice with GBM harbor AIDS-level CD4 counts, as well as contracted, T-cell deficient lymphoid organs, thus underscoring the fact that the T-cell lymphopenia in GBM patients in not treatment-related but rather a characteristic of the disease. It was further determined unexpectedly that “missing” naïve T-cells in GBM patients are found sequestered in large numbers in the bone marrow. In some aspects, sequestration of T-cells in the bone marrow is the result of the loss of S1P1 from the T-cell surface, and is reversible upon precluding S1P1 internalization.

Provided herein are methods for modulating surface display of S1P1 utilizing pathways associated with one or more of S1P1 display and stability, arrestins, and G Protein-Coupled Receptor Kinase 2 (GRK2).

Sphingosine-1-phosphate receptor 1 (S1PR1 or S1P1) is one of five G protein-coupled receptors (GPCR) (S1P1 through 5) that bind the lipid second messenger, sphingosine-1-phosphate (S1P). See NCBI Reference Sequence No. NP 001307659.1. Without being bound by theory, the S1P-S1P1 axis is believed to play a role in lymphocyte trafficking. Naïve T-cell egress from, e.g., bone marrow, may utilize functional S1P1 on the cell surface: In this way, S1P1 serves naïve T-cells as an “exit visa.” A chemotactic S1P1 gradient spanning the blood and bone marrow contributes to T-cell egress from the marrow into the circulation. Disruptions to this gradient result can in T-cell trapping within the marrow and T-cell lymphopenia.

S1P1 is a phosphosphingolipid that is an extracellular ligand for S1P1, and that is believed to play a role in immune cell trafficking and immunomodulation, e.g., through an interaction with S1P1.

Arrestins are a family of proteins believed to play a role in regulating signal transduction of GPCRs, for instance by preventing activation of the GPCR or by linking the GPCR to internalization machinery (e.g., clathrin and/or clathrin adapter AP2).

GRK2 is a GPCR kinase that phosphorylates GPCRs in T-cells, and it is believed that such phosphorylation promotes binding of arrestins (e.g., β-arrestins) to the GCPR.

### Methods of Promoting Surface Display of S1P1 on T-Cells

Provided herein are methods for promoting surface display of S1P1 on T-cells. Such surface display of S1P1 can be promoted by increasing expression of S1P1 on the surface of T-cells. In some aspects, surface display of S1P1 is promoted by stabilizing S1P1 on the surface of T-cells. In some aspects, surface display of S1P1 on T-cells is promoted by inhibiting internalization of the S1P1 by the T-cells. Inhibition of internalization can include targeting S1P1 internalization pathways, including pathways involving arrestins (e.g., β-arrestins), GRK2, clathrin, and/or clathrin adapter AP2.

Thus, some aspects involve administering, to a subject, a S1P1 modulator that reduces β-arrestin recruitment in a T-cell. Some aspects involve administering an effective amount of a β-arrestin inhibitor, such as a β-arrestin 1 inhibitor or a β-arrestin 2 inhibitor, to the subject. In some aspects, the inhibitor is an antagonist, such as a small molecule antagonist. In some aspects, a GRK2 inhibitor is administered to the subject. In some aspects, an inhibitor of clathrin-mediated endocytosis is administered to the subject. In some aspects, a granulocyte colony-stimulating factor is administered to the subject.

Also provided herein are methods of treating diseases or conditions associated with insufficient surface display of S1P1 on T-cells. Exemplary diseases or conditions involve those associated with T-cells sequestered from systemic circulation, for instance via sequestration in bone barrow. Such sequestration can result in a high ratio of sequestered T-cells (e.g., in bone marrow):circulating T-cells. For instance, in some aspects the subject has a bone marrow:blood T-cell ratio of greater than 1, such as about 5:1, about 10:1, about 15:1, or about 20:1. In some aspects, the subject has reduced levels of T-cells in contracted lymphoid organs, such as the lymph nodes, thymus, and/or spleen. In some embodiments the subject has T-cell lymphopenia.

In some aspects, the disease or condition is an intracranial disease or condition, such as an intracranial tumor. In some aspects the disease or condition is a primary intracranial tumor, an intracranial metastatic tumor, inflammatory brain disease or disorder, stroke, or a traumatic brain injury. In some aspects, the disease or condition is glioblastoma.

As already mentioned, T-cell sequestration can impact a variety of diseases or conditions. In some aspects, the sequestered T-cells are naïve T-cells. In some aspects, the sequestered T-cells are CD4+ T-cells. In some aspects, the sequestered T-cells are CD8+ T-cells. In some aspects, T-cells are sequestered while B-cells, NK cells, and/or granulocytes/monocytes are not sequestered.

Also provided are methods of promoting surface display of S1P1 on T-cells in combination with other T-cell activating therapies. Such T-cell activating therapies include, but are not limited to, administering a 41BB agonist and/or a checkpoint blockade (e.g., a PD-1 blockade).

### Pharmaceutical Compositions

Also provided herein are pharmaceutical composition comprising an agent that promotes surface display of S1P1 on a T-cell. The agent can target any of a variety of pathways associated with surface display of S1P1 on the T-cell, including one or more pathways associated with surface expression of S1P1 and S1P1 internalization. In some aspects, the agent stabilizes S1P1 on the surface of the T-cell.

In some aspects, the agent is a S1P1 modulator that reduces β-arrestin recruitment in the T-cell. In some aspects, the agent is a β-arrestin inhibitor, such as a β-arrestin 1 inhibitor or a β-arrestin 2 inhibitor. In some aspects, the inhibitor is an antagonist, such as a small molecule antagonist. In some aspects, the agent is a GRK2 inhibitor. In some aspects, the agent is an inhibitor of clathrin-mediated endocytosis. In some aspects, the agent is a granulocyte colony-stimulating factor.

Pharmaceutical compositions can be formulated in various ways using art-recognized techniques. In some aspects, the pharmaceutical compositions contain a pharmaceutically acceptable carrier. Examples of suitable pharmaceutical composition excipients and formulation methods can be found in Remington's Pharmaceutical Sciences, 20th ed. (Mack Publishing Co., Easton, Pa.). Such formulations may be suitable for administration by various routes, including but not limited to intradermal, intramuscular, intraperitoneal, intravenous, subcutaneous, epidural, and oral routes.

In another aspect, the present disclosure provides compositions comprising one or more of compounds as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

The disclosure further relates to prognostic, diagnostic, theragnostic, and therapeutic methods for diseases or disorders associated with S1P1 loss from the surface of T-cells. The aforementioned compositions and methods also concern related vectors, cells, cell-lines, and animal models. Also provided are articles of manufacture, such as a kit or a packaged system, comprising or related to any of the aforementioned compositions and methods provided by the invention.

### EXAMPLES

The following examples are included as illustrative of the methods and compositions described herein. These examples are in no way intended to limit the scope of the invention. Other aspects of the invention will be apparent to those skilled in the art to which the invention pertains.

### Example 1

T-Cell Lymphopenia and Splenic Contraction in Treatment-Naïve Patients with Glioblastoma.

The records of patients were reviewed at study center hospitals for a period covering the past 10 years to identify patients who met the following criteria: 1) GBM diagnosis; 2) complete blood counts (CBC) at presentation; and 3) CT of the chest/abdomen/pelvis. Lymphocyte counts and splenic volumes were assessed. GBM patient data were compared to all trauma patients evaluated in the emergency department over the same 10-year period fitting the same age range and with a CBC and normal abdominal CT imaging, as determined by a radiologist. Exclusion criteria for both cohorts included history of autoimmune disorder, immune-deficiency, hematologic cancer, splenic injury, active infection, or chemotherapy. Ultimately, 300 patients with GBM and 46 controls satisfied the above inclusion criteria (Table 1): Numbers were not determined a priori. Spleen volumes were determinable in 278 patients and 43 controls; dexamethasone exposure/dosing information was available for 284 patients.

TABLE 1
Retrospective Study: Patient and Control Characteristics

| | | |
|---|---|---|
| | No. of controls (%) | No. of patients (%) |
| Characteristics | N = 46 | N = 300 |

Age

| | | |
|---|---|---|
| Median | 62.5 | 66 |
| Range | 38-83 | 21-91 |

| | | | | |
|---|---|---|---|---|
| Sex | | | | |
| Male | 28 | (61) | 162 | (54) |
| Female | 18 | (39) | 138 | (46) |
| Steroid status | | | | |
| Naive | 46 | (100) | 187 | (63) |
| Experienced | 0 | (0) | 97 | (32) |
| Unknown | 0 | (0) | 16 | 5) |

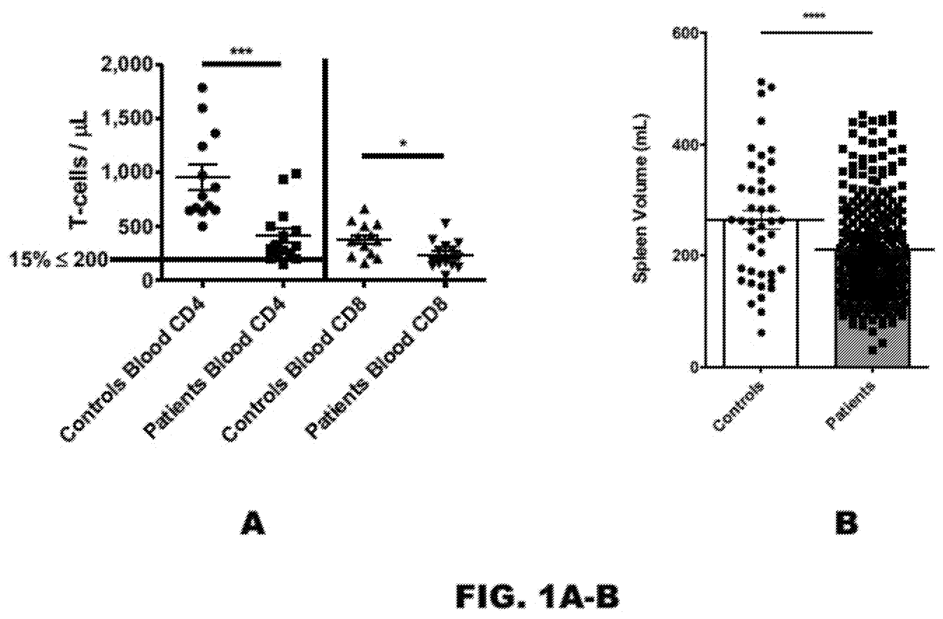

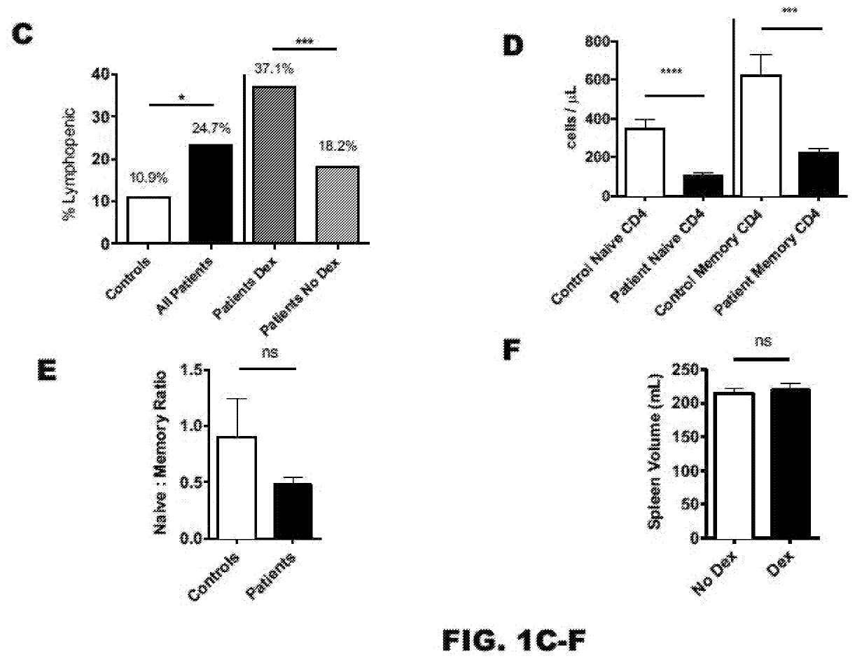

Generalized lymphopenia was present in treatment-naïve GBM patients, with treatment-naïve defined as no prior biopsy, resection, chemotherapy, or radiation. As some patients had been diagnosed at outside hospitals prior to presentation, previous dexamethasone exposure varied. Patients were divided into those entirely dexamethasone-naïve versus those receiving at least a single dose of dexamethasone. Lymphopenia was present in 24.7% of all GBM patients (18.2% of dexamethasone-naïve; 37.1% of dexamethasone-experienced) compared to 10.9% of controls, with lymphopenia defined as lymphocyte count <1000 cells/μL) (FIG. 1C).

To examine T-cell counts specifically, a new cohort of treatment-naïve patients with GBM (n=15), as well as controls meeting similar demographics (n=13) was prospectively studied (Table 2). Patients were dexamethasone-naïve and demonstrated a prevalent, severe reduction in T-cell counts, with a mean CD4 count of 411 cells/μL (control mean 962 cells/μL). CD8 counts were also significantly lower in patients than controls (FIG. 1A). Notably, ˜15% of treatment-naïve GBM patients presented with CD4 counts less than 200 cells/μL, the threshold demarcating AIDS in HIV-infected individuals. T-cell loss trended towards being more severe among naïve T-cells (CD27+CD45RA+) than among memory (CD45RO+), with patients exhibiting decreased ratios of naïve to memory T-cells compared to controls (FIGS. 1D, 1E).

TABLE 2
Prospective Study: Patient and Control Characteristics

| | No. of controls (%) | No. of patients (%) |
|---|---|---|
| Characteristics | N = 13 | N = 15 |
| Age | | |
| Median | 68 | 56 |
| Range | 41-86 | 30-75 |
| Sex | | |
| Male | 7 (54) | 11 (73) |
| Female | 6 (46) | 4 (27) |

Splenic volume was observed to be markedly contracted in GBM patients (32% mean size reduction), with an overall mean of 217.1 milliliters (mL) compared to 317.3 mL in controls (FIG. 1B). Splenic volume in patients was not influenced by dexamethasone exposure (214.4 mL in dexamethasone-naïve; 219.3 mL in dexamethasone-experienced, FIG. 1F).

### Example 2

### Recapitulated T-Cell Lymphopenia and Lymphoid Organ Contraction in Murine Glioma.

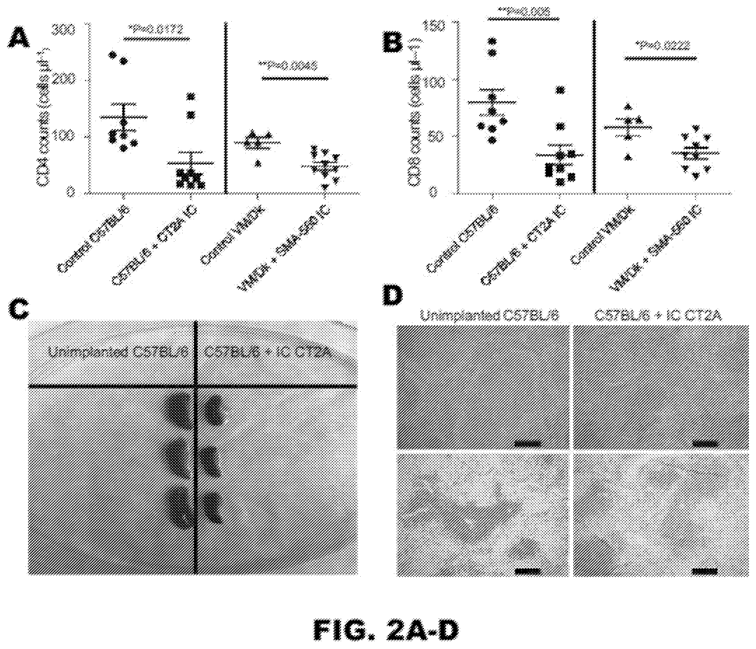

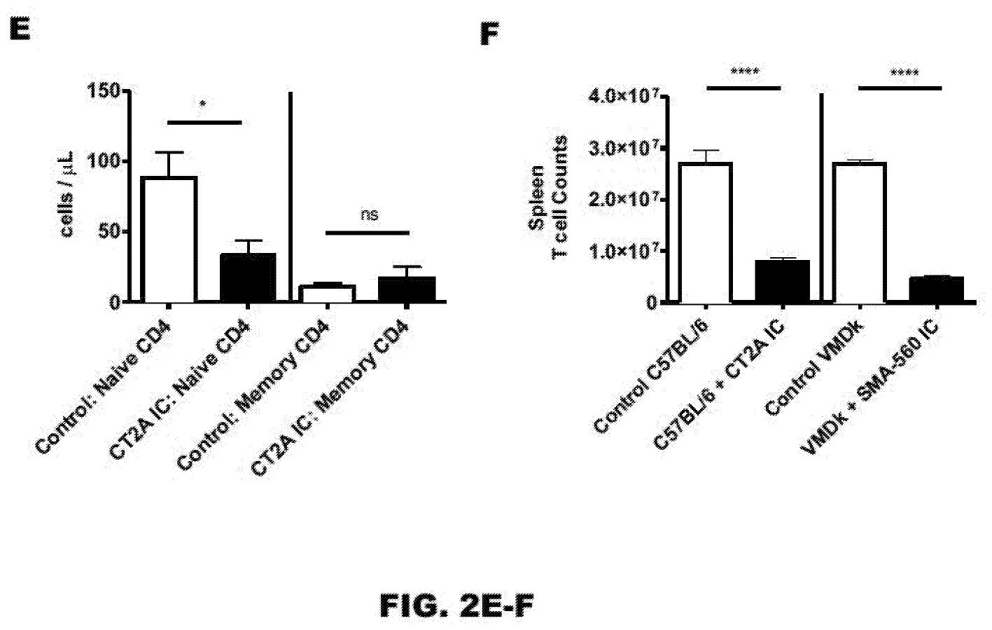

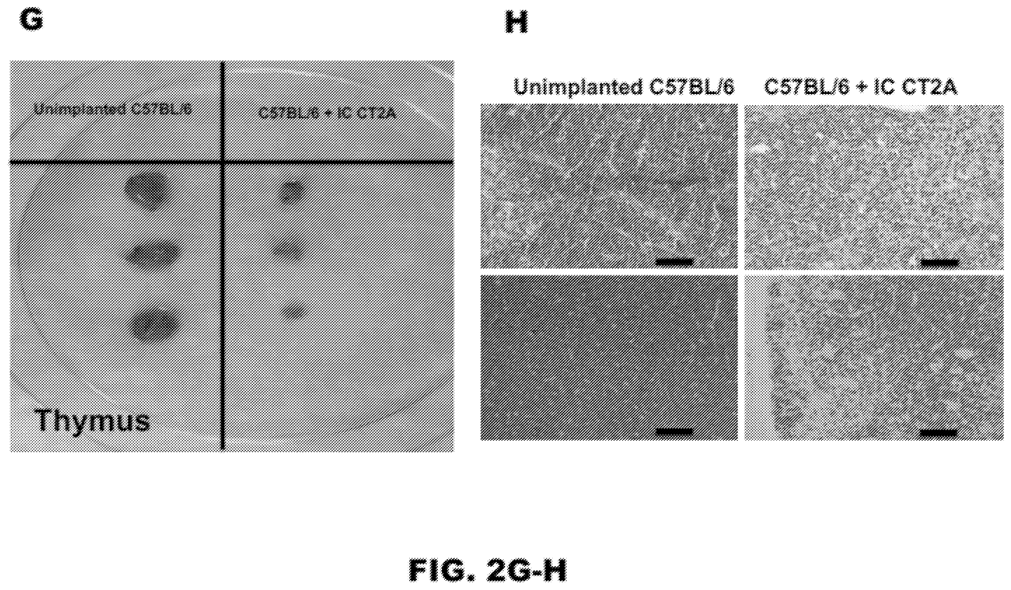

To assess for similar changes in murine glioma models, SMA-560 or CT2A murine glioma cells were implanted stereotactically into the brains (intracranial=IC) of syngeneic VM/Dk or C57BL/6 mice, respectively. Blood, spleen, cervical lymph nodes (CLN), and thymus were analyzed once tumors had become sizeable (Day 18-20). Mice were exclusively treatment-naïve. Both tumor models demonstrated significant T-cell lymphopenia in the CD4 and CD8 compartments (FIGS. 2A, 2B). As with patients, naïve (CD62LhiCD44lo) T-cell numbers were more prominently diminished. Memory (CD44hi) T-cell counts were not significantly reduced (FIG. 2E). The splenic contraction observed in patients with GBM was recapitulated in mice (FIG. 2C), and volume contractions further typified CLN and thymus (thymus depicted in FIG. 2G).

Accompanying the volume reductions in lymphoid organs were significant decreases to organ T-cell counts (spleen counts depicted in FIG. 2F). Histologic examination and immunohistochemical staining of spleen, thymus, and cervical lymph node revealed marked lymphodepletion, primarily in T-cell-dependent areas. Lymphoid necrosis was also present (FIGS. 2D, 2H). Severe T-cell disappearance thus appeared systemic, characterizing both blood and lymphoid organs.

### Example 3

Naïve T-Cells Accumulate in the Bone Marrow of Mice and Patients with GBM

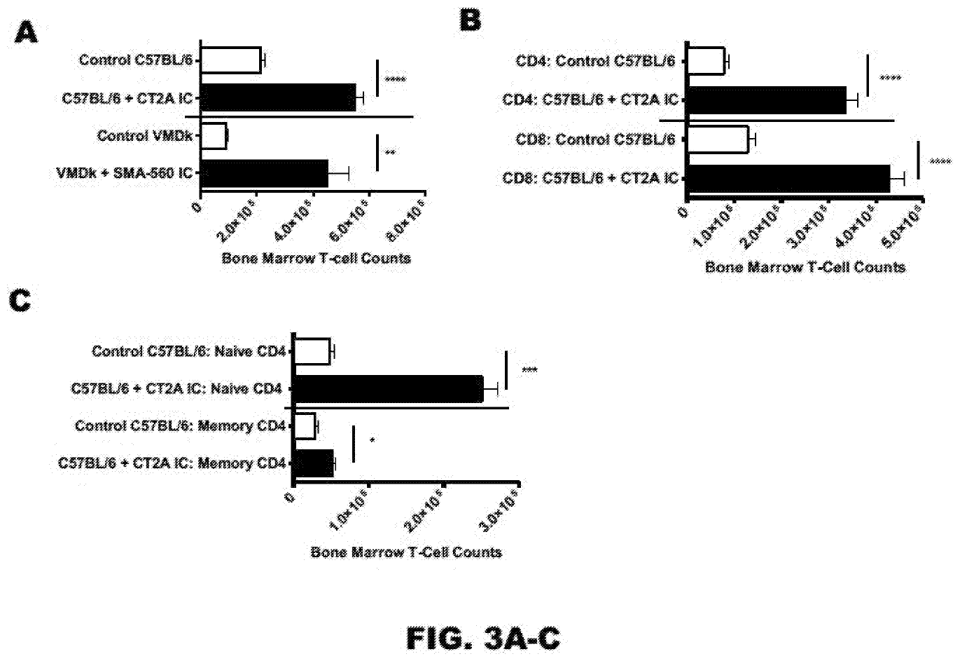

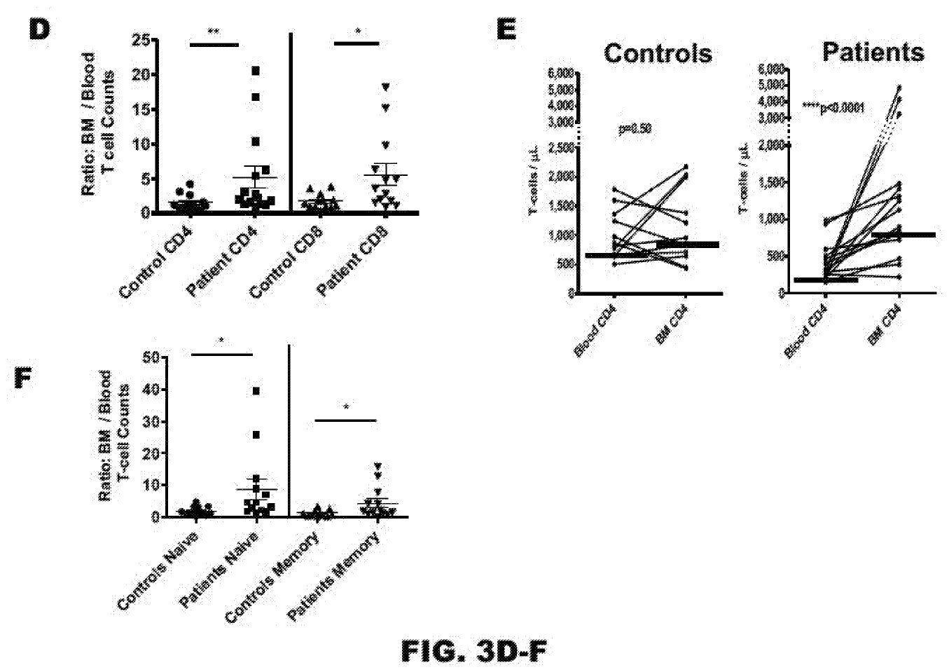

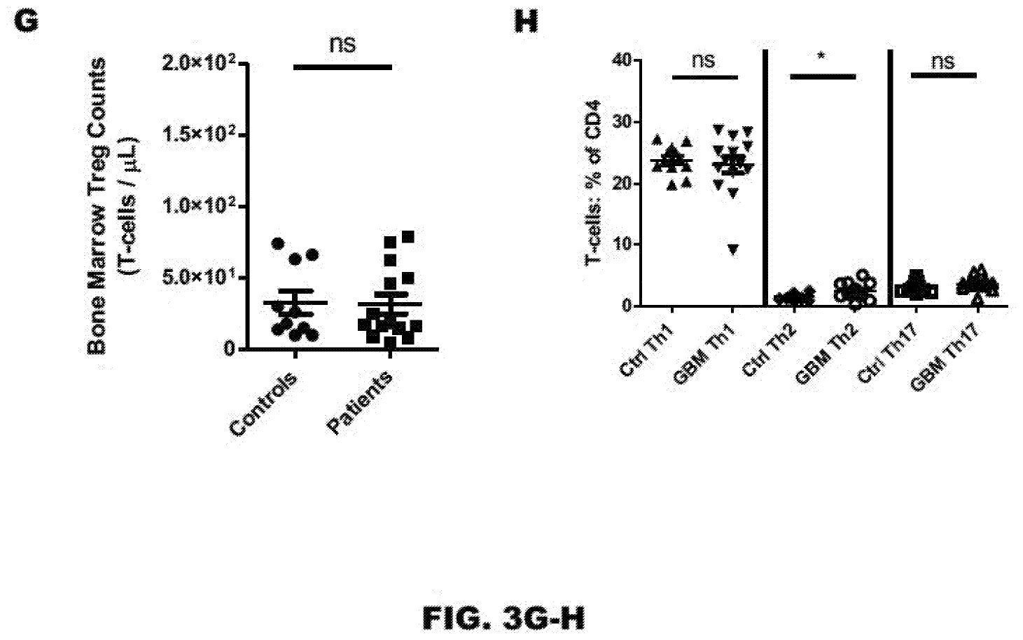

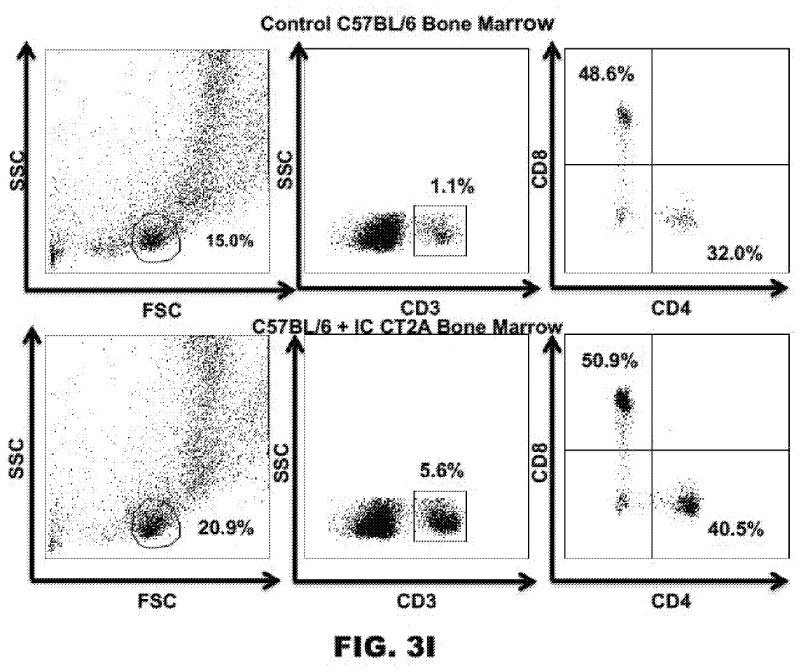

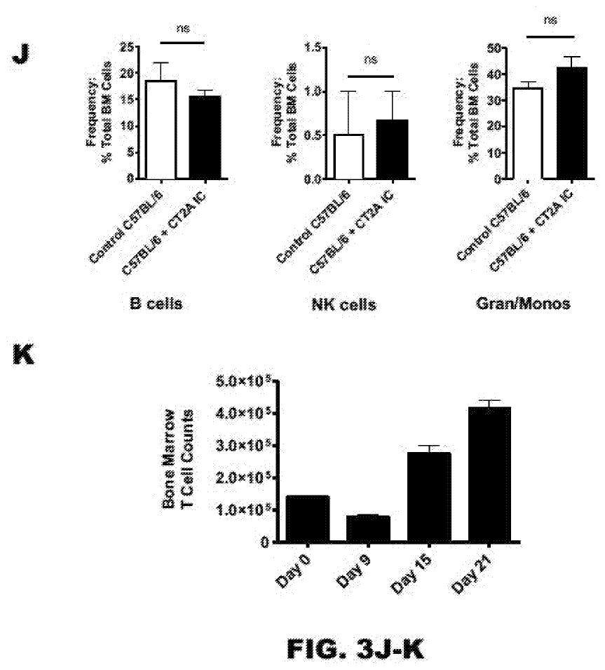

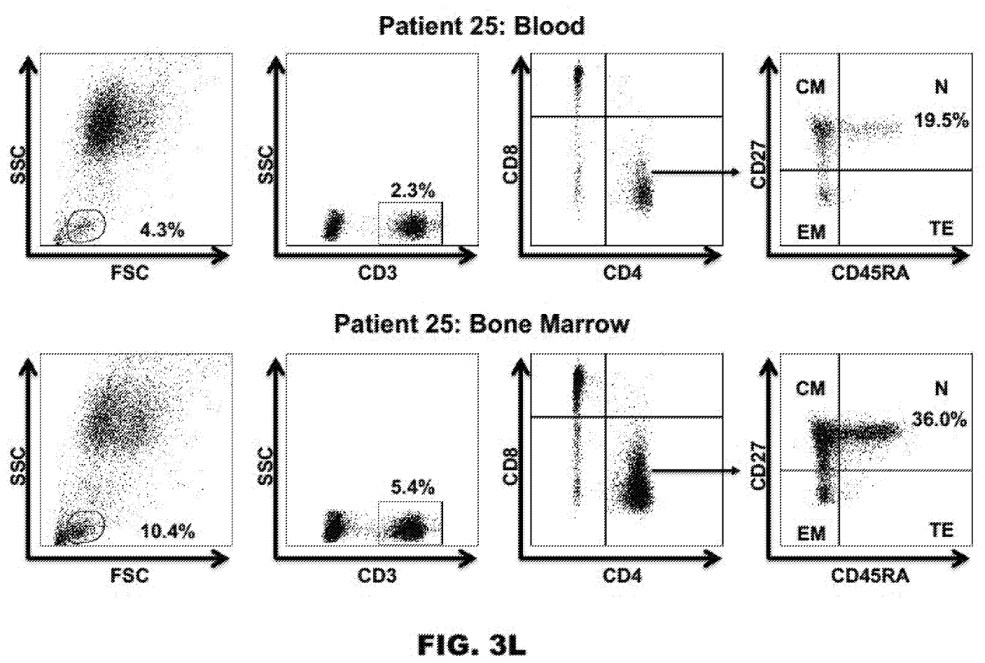

Diminished naïve T-cell counts suggested deficient production, leading to the investigation of the bone marrow of glioma-bearing mice for T-cell progenitor frequencies. This analysis instead revealed that naïve T-cell disappearance from blood and lymphoid organs was met conversely with 3- to 5-fold expansions of mature, single-positive T-cell numbers within the bone marrow of mice bearing either SMA-560 or CT2A IC (FIG. 3A; sample flow cytometry in FIG. 3I). Immune cell accumulation in the bone marrow was T-cell-specific, with no increases observed for NK-cells, B-cells, or granulocytes/monocytes (FIG. 3J). Both CD4+ and CD8+ T-cells accumulated (FIG. 3B), albeit disproportionately those with a naïve phenotype (CD44loCD62Lhi) (FIG. 3C). A time course for T-cell accumulation is provided in FIG. 3K.

It was investigated whether this finding it was mirrored in patients with GBM. Blood and bone marrow aspirates were collected from 15 treatment-naïve GBM patients and 15 healthy controls undergoing spinal fusion (from whom bone marrow aspirates are often collected intra-operatively for employment in fusion constructs). All bone marrow was harvested from patients and controls following the induction of general anesthesia for their respective surgeries (resection or fusion). Aspirates were collected from the iliac crest prior to incision or to administration of any indicated intra-operative steroids. Samples were analyzed by flow cytometry.

In patients with GBM, a significant re-allocation of T-cells to bone marrow, as compared to blood, was uncovered. While bone marrow T-cell counts varied widely among all individuals, the controls typically had matching T-cell counts across bone marrow and blood (median marrow to blood ratio for CD4+ T-cells 1.06:1; for CD8+ T-cells 1.42:1). This homeostasis was disrupted in GBM patients, who nearly universally had higher T-cell counts in their bone marrow, with marrow to blood ratios ranging as high as 20:1 (FIG. 3D). In GBM patients, there was a consistent increase in both CD4 and CD8 counts as one moved from blood to bone marrow (p<0.0001 and p=0.0007, respectively, Wilcoxon matched-pairs signed rank test; CD4+ T-cells depicted in FIG. 3E). Indeed, 14 of 15 GBM patients had higher T-cell counts in bone marrow than in blood, while for controls this was true in only 8 of 15, (p=0.01, Chi Square analysis). As with mice, naïve (CD27+CD45RA+) T-cells were over-represented in the bone marrow (CD4+ T-cells depicted in FIG. 3F, sample flow cytometry depicted in FIG. 3L). Exploring CD4 subsets, no difference was found in the counts of bone marrow Tregs across patients and controls (FIG. 3G). Although most T-cells detected in marrow were naïve, differentiated CD4+ helper T-cell (Th1, 2, or 17) subsets were analyzed finding no substantial differences in the relative representation of each across the bone marrow of patients and controls (FIG. 3H).

### Example 4

T-Cell Accumulation in Bone Marrow Reflects Intracranial Tumor Location Rather than Tumor Histologic Type

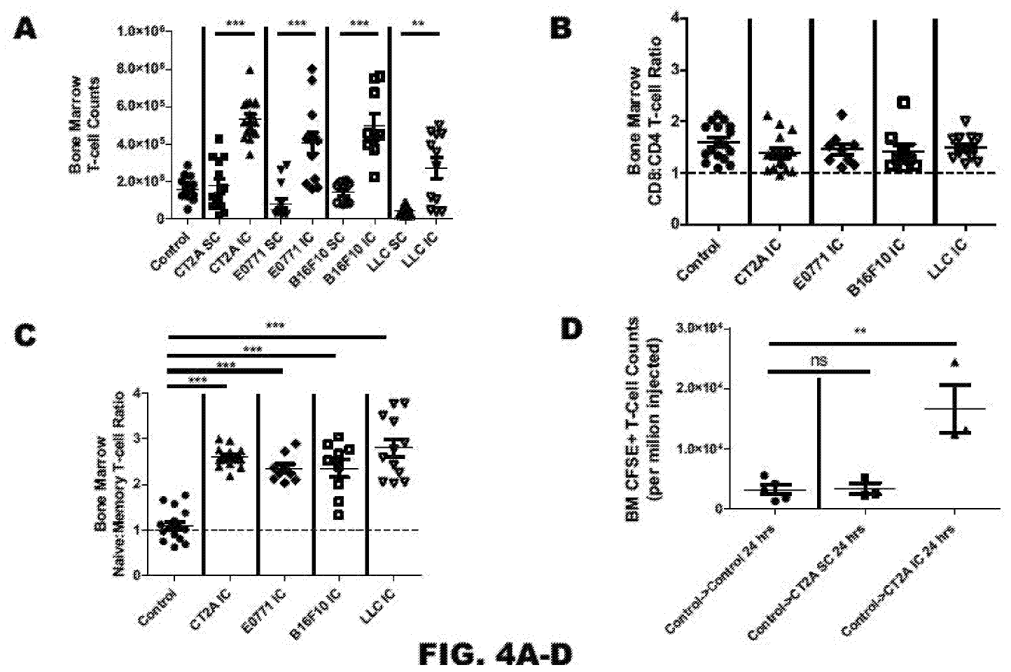

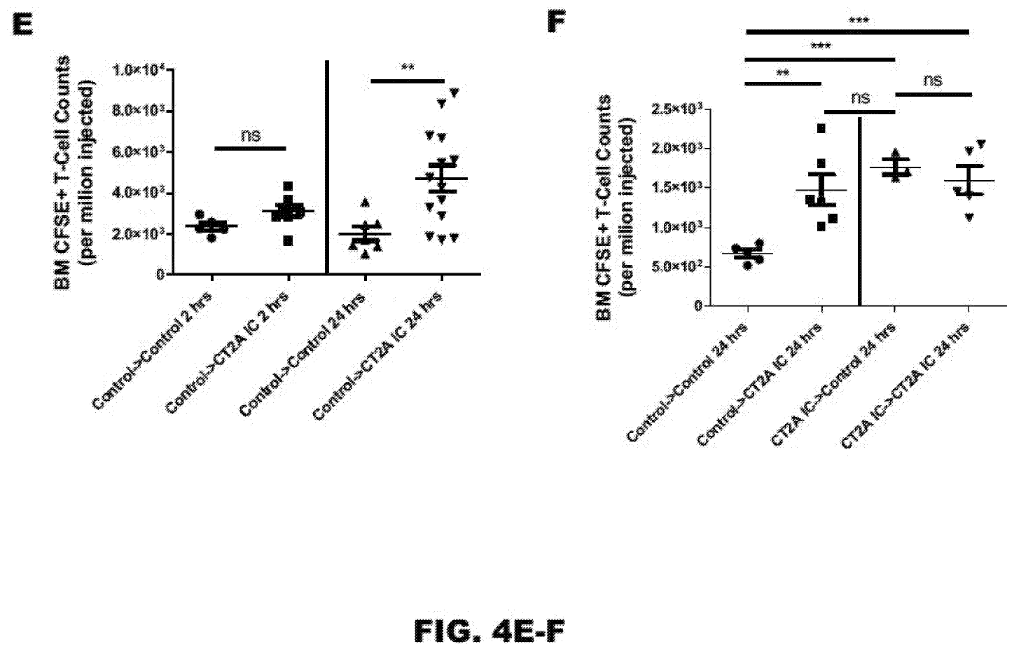

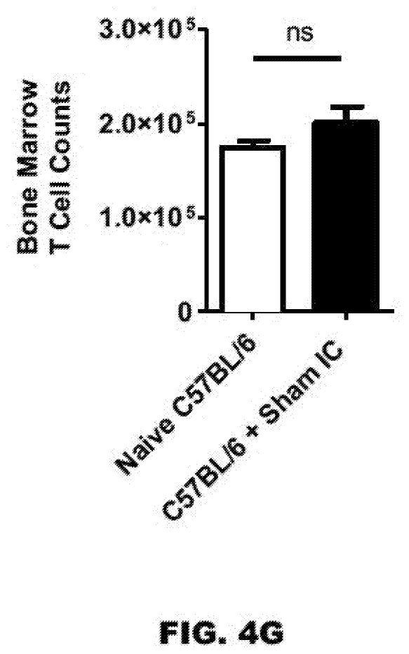

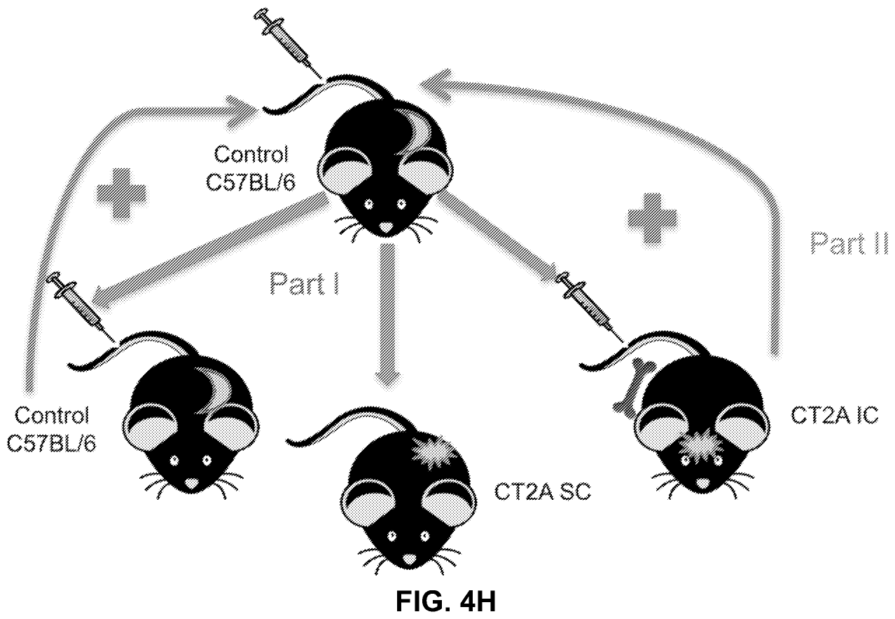

Whether accumulation of T-cells in bone marrow characterized cancer more generally or, rather, was specific to either glioma or the intracranial tumor environment was investigated. To test this, E0771 breast carcinoma, B16F10 melanoma, Lewis lung carcinoma (LLC), or CT2A gliomas were each implanted either IC or subcutaneously (SC) into syngeneic C57BL/6 mice and bone marrow T-cell frequency assessed. Notably, each IC tumor provoked significant accumulation of T-cells in bone marrow, regardless of the primary tumor type. Conversely, none of the SC-situated tumors, including glioma, evoked the same phenomenon (FIG. 4A). Control IC injections with saline and methycellulose produced no increase in bone marrow T-cell numbers (FIG. 4G). CD4+ and CD8+ T-cells accumulated in the bone marrow in approximately equal proportions across all tumor types (FIG. 4B), but for all models, accumulating T-cells were disproportionately naïve (CD44loCD62Lhi) (FIG. 4C).

The accumulation of largely naïve T-cells in the bone marrow indicated homing or sequestration. It was therefore investigated whether adoptively transferred naïve T-cells would likewise preferentially collect in the bone marrow of glioma-bearing mice. Naïve C57BL/6 spleens were harvested as a source of donor leukocytes. Cells (1×107) were CFSE-labeled and injected via tail vein into naïve control mice or mice bearing CT2A glioma IC or SC. At 24-hours, analysis revealed increased numbers of labeled T-cells in the bone marrow uniquely in hosts bearing CT2A IC, and not in hosts bearing CT2A SC (FIG. 4D). This experiment was repeated with IC CT2A recipient mice, assessing at time-points 2- and 24-hours post-transfer. Although present again in marrow at 24-hours, labeled T-cells did not to accumulate in the bone marrow at the 2-hour time point, suggesting T-cell trapping or sequestration rather than direct bone marrow homing (FIG. 4E).

As a crossover, T-cells that had accumulated within the bone marrow of glioma-bearing mice were harvested, enriched, labeled with CFSE, and injected into tail veins of naïve control mice. T-cells that had accumulated in the bone marrow of glioma-bearing mice re-accumulated within the marrow of naïve mice with equivalent efficiency. Transferring the same cells into tumor-bearing hosts yielded no further increase in marrow accumulation (FIG. 4F). These experiments indicated that the acquisition of T-cell phenotypic changes precipitate their sequestration, as compared to changes to the bone marrow itself (schematic in FIG. 4H).

### Example 5

Loss of Surface S1P1 on T-Cells Directs their Sequestration in Bone Marrow in the Setting of Intracranial Tumor

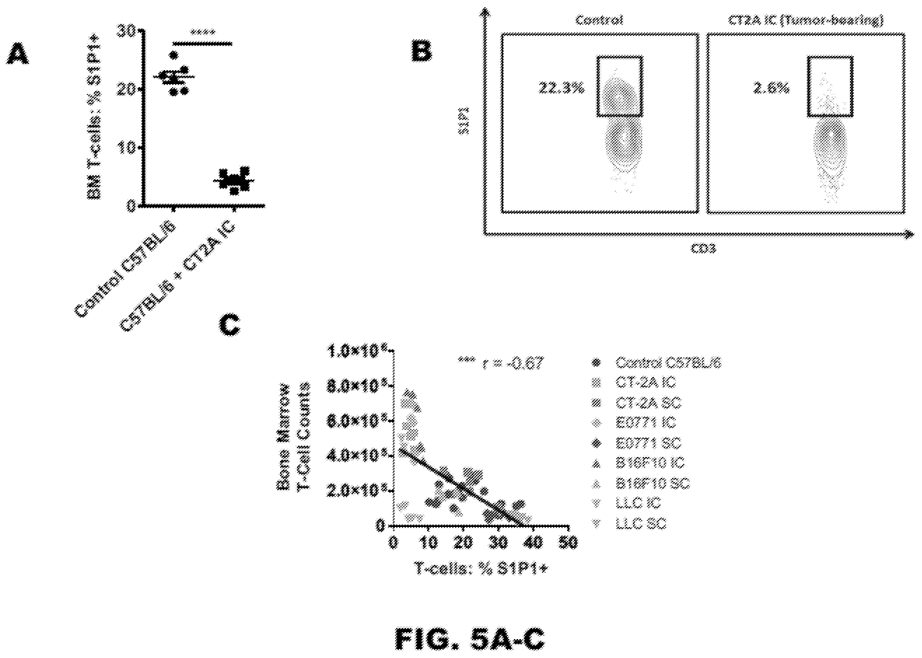

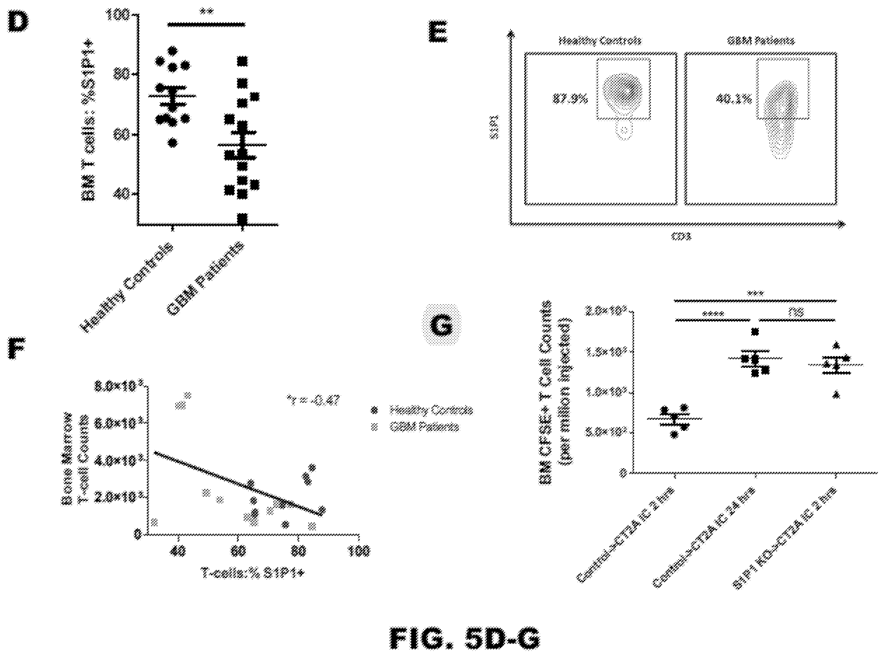

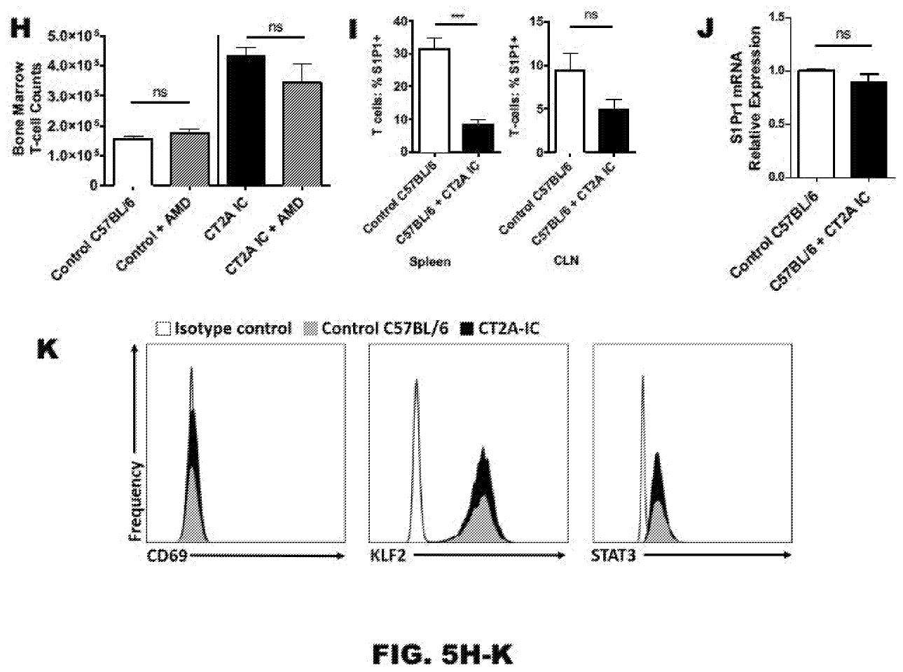

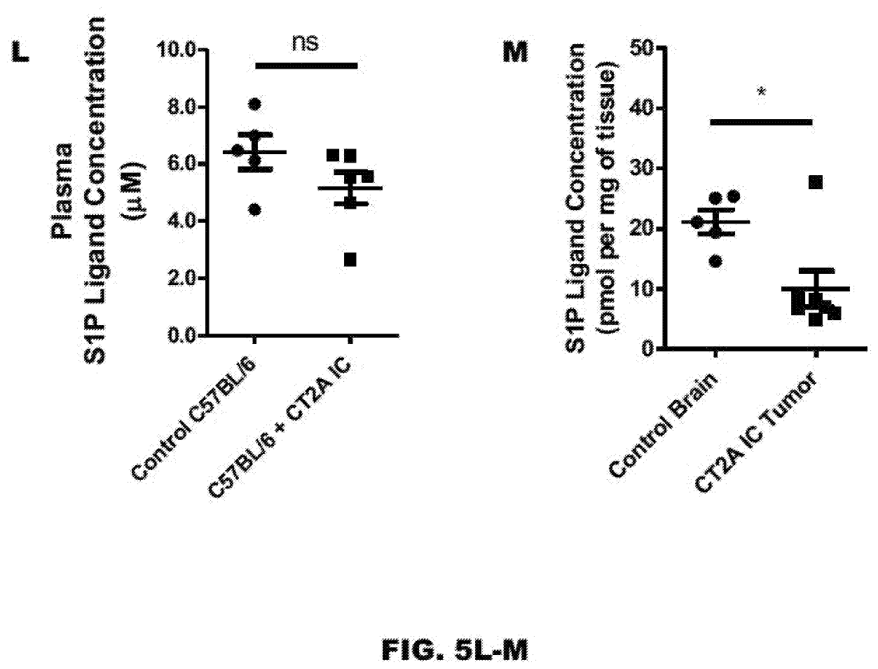

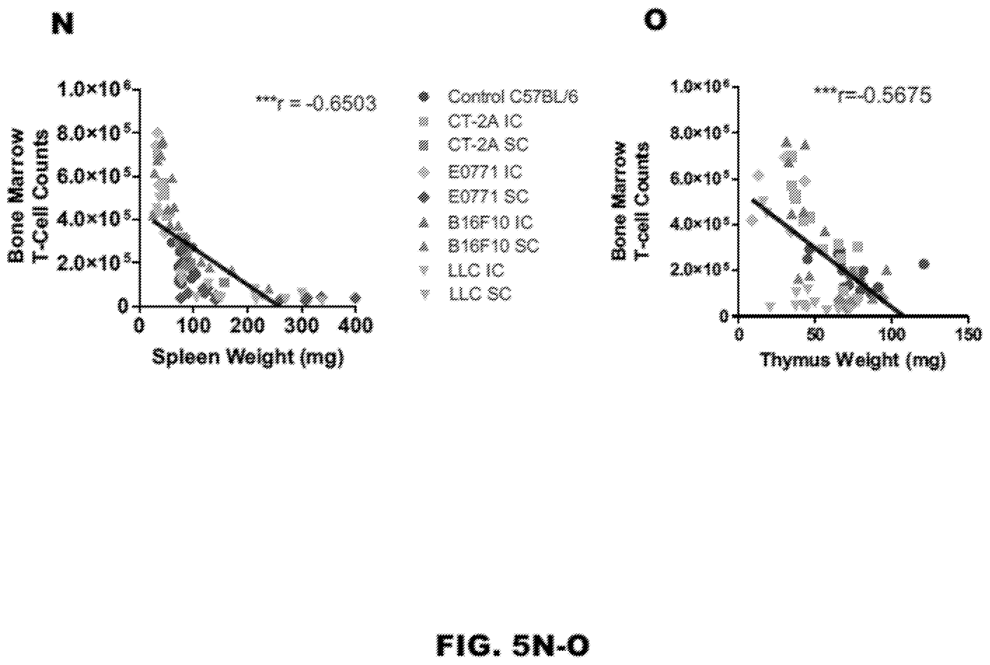

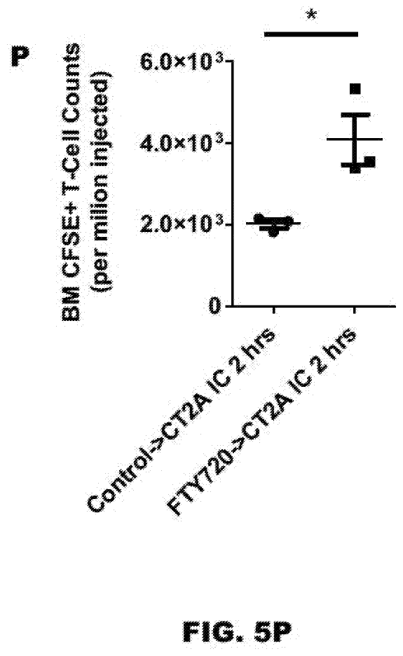

As indicated by FIGS. 4D-F, T-cells acquire alterations facilitating their sequestration in the glioma-bearing state. It was investigated whether the relevant alteration might be diminished levels of surface S1P1 (previous investigation of the CXCR4-CXCL12 axis did not show to find a relationship) (FIG. 5H).

Surface S1P1 levels were assessed on T-cells in the bone marrow of control mice and mice bearing IC CT2A glioma. For the detection of otherwise fleeting S1P1 on the cell surface by flow cytometry, harvested tissues were immediately placed into a fixative solution to cross link surface molecules, in which no solutions contained fetal calf serum in order to avoid ligand-induced internalization. Mice with IC CT2A demonstrated markedly reduced T-cell S1P1 levels in bone marrow (FIGS. 5A, 5B) and moderately reduced T-cell S1P1 levels in contracted spleens and CLN (FIG. 5I).

Without being bound by theory, it is believed that loss of S1P1 might result from changes to gene expression or from alterations at the protein level (e.g., increased receptor internalization or decreased recycling). To assay for altered S1pr1 expression (the gene encoding S1P1), qRT-PCR of T-cells sorted from the spleens of control and glioma-bearing mice was performed. No differences in S1pr1 transcript numbers were detected (FIG. 5J). Likewise, RNA flow cytometry of T-cells revealed no differences in levels of the upstream S1pr1 modulators CD69, KLF2, or STAT3 (FIG. 5K).

As S1P1 receptor loss or internalization might accompany increased levels of S1P1 ligand, S1P1 concentrations in the plasma and tumors of control and glioma-bearing mice were assessed by liquid chromatography-tandem mass spectrometry (LC-MS/MS). No differences were seen in the plasma, and IC CT2A gliomas instead showed slightly decreased levels of S1P1 compared to normal brain (FIGS. 5L, 5M).

Next, an association between T-cell S1P1 levels and their sequestration in bone marrow across various IC and SC tumor models was investigated. A strong inverse relationship was uncovered between T-cell S1P1 levels and T-cell numbers in bone marrow (FIG. 5C). Furthermore, bone marrow T-cell sequestration was associated with the presence of contracted spleens and thymuses (FIGS. 5N, 5O), indicating lymphoid organ contraction may be contextually important.

Flow cytometry was used to explore whether similar alterations in S1P1 were present in the bone marrow of patients with GBM. The results paralleled the findings in the murine models, with GBM patients exhibiting decreased levels of S1P1 on the T-cell surface compared to healthy, age-matched controls (FIGS. 5D, 5E). Likewise, an inverse relationship emerged between bone marrow T-cell counts and surface S1P1 levels across GBM patients and controls (FIG. 5F).

Given these associations, it was subsequently investigated whether forced loss of surface S1P1 on T-cells might be sufficient to facilitate their sequestration. As shown in FIGS. 4D and 4F, transferred T-cells accumulated in the bone marrow of IC glioma-bearing mice after 24-hours. This accumulation had not yet occurred at 2-hours following transfer, which would have been a proxy for active T-cell homing to marrow. Thus, without being bound by theory it is believed that the 24-hour delay was a function of the time during which T-cells lose surface S1P1 when transferred into glioma-bearing recipients; and that T-cells with prior loss of surface S1P1 would be subject to more immediate sequestration in mice bearing glioma.

With this in mind, S1P1 conditional knockout (KO) mouse were employed in further investigations. In particular, mice with loxP sites flanking exon 2 of S1pr1 were crossed with mice possessing inducible Cre recombinase. When treated with tamoxifen, these mice demonstrated a decrease in S1P1 protein levels. Donor splenocytes were harvested from tamoxifen-treated S1P1-KO mice and labeled with CSFE. The splenocytes were injected via tail vein into IC CT2A-bearing recipients, and accumulation in bone marrow assessed at 2- and 24-hours post-injection. T-cells from S1P1-KO mice accumulated in the bone marrow within 2-hours, while cells from WT C57BL/6 (control) donors did not (FIG. 5G). Similar results were obtained when S1P1 loss was instead imposed pharmacologically by treating recipient mice with the S1P1 functional antagonist FTY720 at the time of adoptive transfer (FIG. 5P).

### Example 5

### Hindering S1P1 Internalization Abrogates T-Cell Sequestration in Bone Marrow

It was next examined whether increased/stabilized surface S1P1 might abrogate bone marrow T-cell sequestration in glioma-bearing mice. An S1P1 “knock-in” (S1P1-KI) mouse strain was used, in which lymphocyte S1P1 internalization is hindered (B6.129P2-S1pr1tm1.1Cys/J), resulting in stabilized cell surface receptor levels. The S1P1 receptor in these mice has disrupted serine residues on the intracellular domain, precluding GRK2 phosphorylation, β-arrestin recruitment, and clathrin-mediated endocytosis.

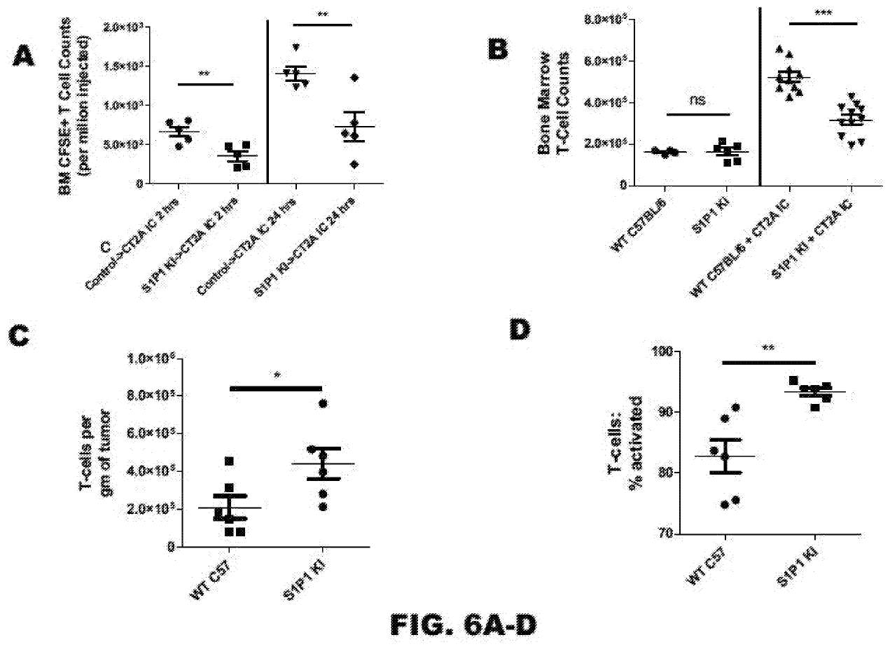

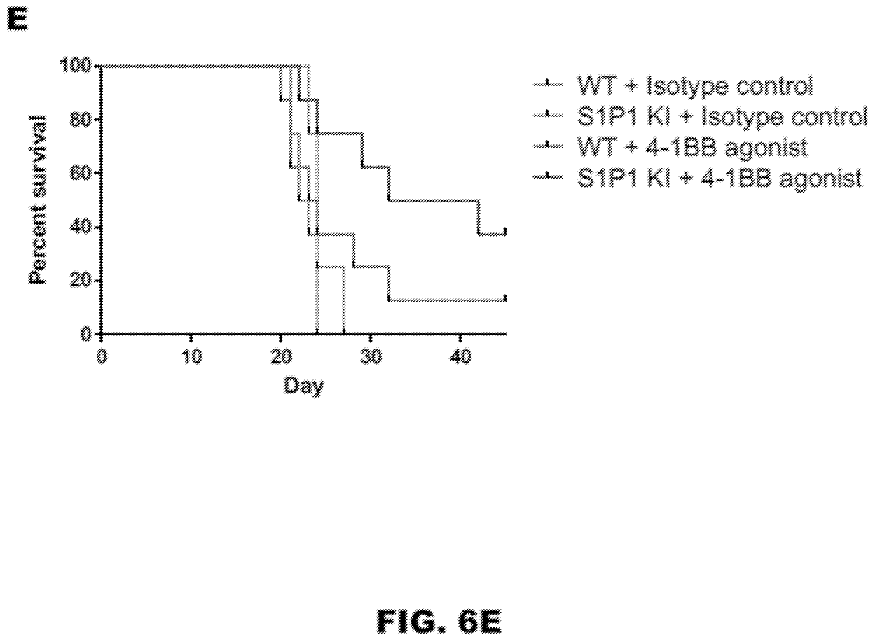

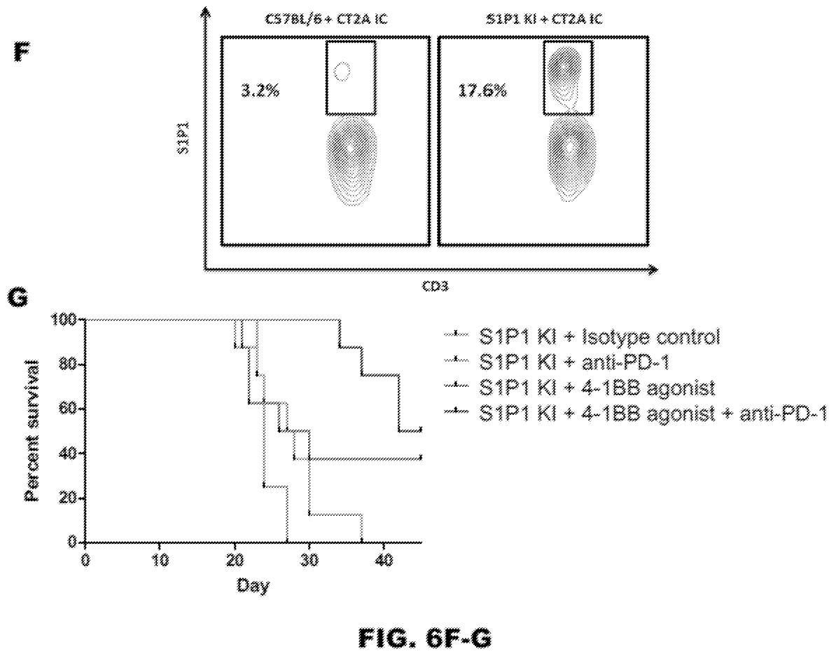

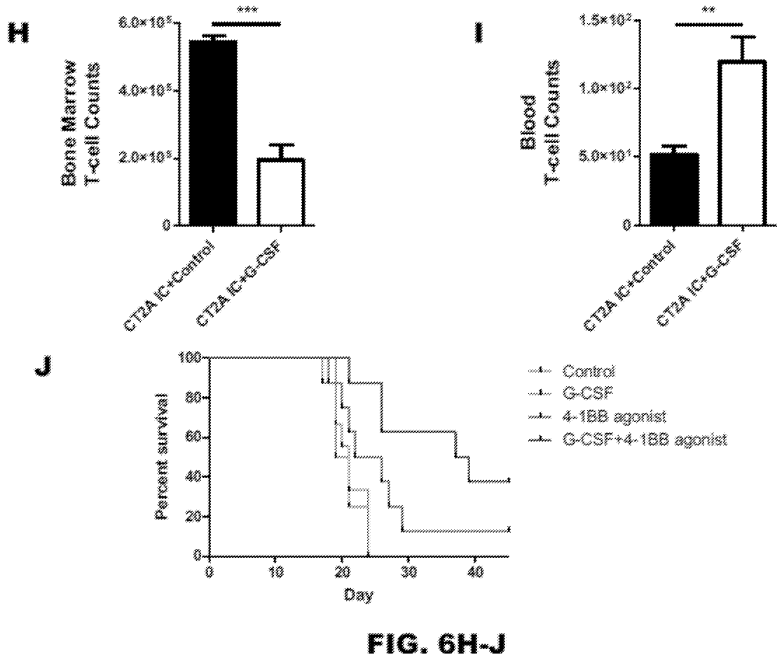

It was tested whether T-cells possessing stabilized, internalization-deficient S1P1 would resist sequestration when adoptively transferred into glioma-bearing mice. Recipient mice were C57BL/6 mice bearing IC CT2A. Donor T-cells were harvested from WT or S1P1-KI mice, CSFE-labeled, and injected IV. Bone marrow of recipient mice was analyzed at 2- and 24-hours post-transfer. At both time-points, T-cells from S1P1-KI donors did not become appreciably sequestered within bone marrow when compared to T-cells from WT donors (FIG. 6A). Likewise, S1P1-KI mice themselves directly implanted with IC CT2A proved similarly resistant to bone marrow T-cell sequestration (FIG. 6B).

IC CT2A tumors from both WT and S1P1-KI glioma-bearing mice were examined to determine whether T-cells “liberated” from sequestration by S1P1 stabilization would travel to the intracranial compartment and effect an anti-tumor response. TIL were analyzed by flow cytometry and their number and phenotype characterized. Tumors from S1P1-KI mice contained higher numbers of CD3+ TIL than those from WT mice (FIG. 6C). Likewise, CT2A-bearing S1P1-KI mice demonstrated increased proportions of CD3+ TIL possessing an activated, effector CD44hiCD62Llo phenotype (FIG. 6D).

Despite displaying higher numbers of activated TIL, tumor-bearing S1P1-KI mice that underwent no further intervention did not consistently show improved survival. S1P1-stabilized (KI) mice treated with a 4-1BB agonist demonstrated improved survival compared to the effects seen with either stabilized S1P1 or with 4-1BB agonism in WT mice alone (FIG. 6E). Representative flow cytometry plot depicting the frequency of S1P1 on the surface of T-cells in the bone marrow of C57BL/6 mice and S1P1 KI bearing IC CT2A tumor is shown in FIG. 6F. Furthermore, in S1P1-KI mice themselves, whereas PD-1 blockade was ineffectual as monotherapy, the effects of 4-1BB agonism and checkpoint blockade proved additive, with the combination prolonging median survival and producing a 50% long-term survival rate (FIG. 6G). Thus, coupling S1P1 stabilization to T-cell activating therapies, such as 4-1BB agonism and/or checkpoint blockade, have a synergistic effect, licensing the anti-tumor capacities of the newly freed T-cells.

Alternative translatable means for freeing sequestered T-cells were explored, and it was uncovered that treating CT2A glioma-bearing mice with G-CSF decreased bone marrow T-cell counts and reversed T-cell lymphopenia (FIG. 6H, 6I). As with S1P1 stabilization alone, G-CSF monotherapy did not to consistently impact survival. When combined with 4-1BB agonism, however, a similar additive effect was achieved, yielding approximately 40% long-term survival. (FIG. 6J).

### Example 6

### Protocols and Methods for Results Described in Examples 1-5.

### Clinical Studies and Specimen Processing

All studies were conducted with approval from the Massachusetts General Hospital Cancer Center Institutional Review Board. For prospective studies, 15 treatment-naïve GBM patients and 15 healthy age-matched controls undergoing spinal fusion were included in the prospective collection of whole blood and bone marrow aspirates. Bone marrow aspirates were collected under general anesthesia from the iliac crest. Using a 14-gauge needle, a total volume of 5 mL was collected. Both blood and bone marrow specimens were collected into purple top, EDTA-containing tubes. Blood and bone marrow were stored at room temperature and processed within 12-hours. Samples were labeled directly with antibodies for use in flow cytometry, and red blood cells subsequently lysed using eBioscience RBC lysis buffer (eBioscience, San Diego, Calif.). Cells were washed, fixed, and analyzed on an LSRII FORTESSA flow cytometer (BD Biosciences).

### Reagents

For human studies, fluorochrome-conjugated antibodies to CD3 (Cat #557705, Clone: SP34-2, Lot #5352959, 1:20; Cat #558117, Clone: UCHT1, Lot #3186876, 1:100; Cat #557851, Clone: SK7, Lot #3193549), CD4 (Cat #558116, Clone: RPA-T4, Lot #6224744, 1:100; Cat #557695, Clone: RPA-T4, 1:20), CD8 (Cat #565310, Clone: SK1, Lot #7003689, 1:20; Cat #557746, Clone: RPA-T8, Lot #79151, 1:20; Cat #558207, Clone: RPA-T8, 1:100), CD45RO (Cat #563722, Clone: UCHL1, Lot #7096923, 1:20), CD25 (Cat #562403, Clone: M-A251, Lot #7088762, 1:20), CD27 (Cat #558664, Clone: M-T271, Lot #7136657, 1:5), CD127 (Cat #563225, Clone: HIL-7R-M21, Lot #7012862, 1:20), CCR6 (Cat #559562, Clone: 11A9, Lot #7019800, 1:100), CCR7 (Cat #557648, Clone: 3D12, Lot #3186974, 1:20), and CXCR4 (Cat #560669, Clone: 12G5, 1:20) were obtained from BD Biosciences (San Diego, Calif.). Antibodies to human CD45RA (Cat #304128, Clone: HI100, 1:20) and CXCR3 (Cat #353738, Clone: G025H7, Lot #B228065, 1:100) were obtained from BioLegend (San Diego, Calif.). Antibodies to human S1P1 (Cat #50-3639-42, Clone: SW4GYPP, Lot #4299074, 1:20) were obtained from eBioscience (San Diego, Calif.). For murine studies, fluorochrome-conjugated antibodies to CD3 (Cat #557666, Clone: 145-2C11, Lot #7096805, 1:100; Cat #553066, Clone: 145-2C11, Lot #7150784, 1:100), CD4 (Cat #553049, Clone: RM4-5, Lot #4189673, 1:100; Cat #558107, Clone: RM4-5, 1:100), CD8 (Cat #551162, Clone: 53-6.7, Lot #4275549, 1:100; Cat #563234, Clone: 53-6.7, Lot #7047617, 1:100), CD44 (Cat #562464, Clone: IM7, Lot #6205542, 1:100; Cat #559250, Clone: IM7, Lot #25892, 1:100), CD62L (Cat #553152, Clone: MEL-14, Lot #40865, 1:100), NK1.1 (Cat #553164, Clone: PK136, Lot #80219, 1:100), B220 (Cat #558108, Clone: RA3-6B2, Lot #6175996, 1:100), and GR-1 (Cat #553128, Clone: RB6-8C5, Lot #09439, 1:100) were obtained from BD Biosciences (San Diego, Calif.). Antibodies to murine S1P1 (Cat #FAB7089A, Clone: 713412, Lot #ACNG0216051, 1:10) were obtained from R&D systems (Minneapolis, Minn.). Probes for RNA PrimeFlow for mouse CD69, KLF2, and STAT3 were obtained from Life Technologies (Carlsbad, Calif.). For qRT-PCR, total RNA was isolated by RNeasy Mini Kit (Cat #74104) from Qiagen (Germantown, Md.). The assays were performed with Mouse S1P1 TaqMan (Cat #Mm02619656_s1) and Mouse GAPDH TaqMan (Cat #Mm03302249_g1) from ThermoFisher (Waltham, Mass.). In vivo therapeutic antibodies (anti-mouse PD-1 (Cat #BE0146, clone: RMP 1-14, Lot #640517M2) and 4-1BB agonist antibody (Cat #BE0169, clone: LOB12.3, Lot #647417M1)) were obtained from Bio-X-cell (West Lebanon, N.H.).

### Mice

Female C57BL/6, VM/Dk, and B6.129P2-S1pr1tm1.2Cys/J S1P1-KI mice were used at 6-12 weeks of age. The generation of B6.129P2-S1pr1tm1.2Cys/J (S1P1-KI) mice has been described previously. S1P1-KI mice carry a Thr-Ser-Ser (TSS) to Ala-Ala-Ala (AAA) mutation in the C-terminus (the last 12 amino acids) of the sphingosine-1-phosphate receptor 1 (S1P1). This mutation leads to a loss in sensitivity for ligand-mediated receptor down-modulation, leading to the partial block in the desensitization process, resulting in resistance to S1P-mediated S1P1 internalization in naïve T-cells. Parental transgenic mice were acquired from the Jackson Laboratory (Bar Harbor, Me.) with in-house breeding colony expansion. C57BL/6 mice purchased from Charles River Laboratories (Wilmington, Mass.) were used as wild-type controls. S1P1 conditional knockout mice were created by crossing B6.12956(FVB)-S1pr1tm2.1Rlp/J, which contains loxP sites flanking exon 2 of S1pr1 gene (JAX Stock #019141), with B6.Cg-Tg(UBC-cre/ERT2)1Ejb/1J (JAX Stock #007001), which contains tamoxifen-inducible Cre. These two mice were obtained from the Jackson Laboratory (Bar Harbor, Me.) and crossed and then back-crossed to obtain mice with the genotype flox/flox Cre (+/−). The mice were then treated with tamoxifen to induce recombination. VM/Dk mice were bred and maintained as a colony at Duke University. Animals were maintained under specific pathogen-free conditions at Cancer Center Isolation Facility (CCIF) of Duke University Medical Center. All experimental procedures were approved by the Institutional Animal Care and Use Committee.

### Cell Lines

Cell lines studied included murine SMA-560 malignant glioma, CT-2A malignant glioma, E0771 breast medullary adenocarcinoma, B16F10 melanoma, and Lewis Lung Carcinoma (LLC). SMA-560 cells are syngeneic on the VM/Dk mouse background, while all others are syngeneic in C57BL/6 mice. SMA-560, CT-2A, B16F10, and LLC cells were grown in vitro in Dulbecco's Modified Eagle Medium (DMEM) with 2 mM 1-glutamine and 4.5 mg/mL glucose (Gibco) containing 10% fetal bovine serum (Gemini Bio-Products). E0771 cells were grown in vitro in RPMI 1640 (Gibco) containing 10% fetal bovine serum plus 1% HEPES (Gibco). Cells were harvested in the logarithmic growth phase. For intracranial implantation, tumor cells in PBS were then mixed 1:1 with 3% methylcellulose and loaded into a 250 μL syringe (Hamilton, Reno, Nev.). The needle was positioned 2 mm to the right of the bregma and 4 mm below the surface of the skull at the coronal suture using a stereotactic frame. 1×10⁴ SMA-560, CT-2A, E0771, and LLC cells or 1×10³ B16F10 cells were delivered in a total volume of 5 μL per mouse. For subcutaneous implantation, 5×10⁵ SMA-560, CT-2A, E0771, and LLC cells or 2.5×10⁵ B16F10 cells were delivered in a total volume of 200 μL per mouse into the subcutaneous tissues of the left flank. All cell lines have been authenticated by using NIST published species-specific STR markers to establish genetic profiles. Interspecies contamination check for human, mouse, rat, African green monkey and Chinese hamster was also performed for each cell line. All cell lines have been tested negative for Mycoplasma spp. and karyotyped, and none are among the ICLAC database of commonly misidentified cell lines. The CellCheck Mouse Plus™ cell line authentication and Mycoplasma spp. testing services were provided by IDEXX Laboratories (Westbrook, Me.).

### Murine Tissue Harvest

Spleen, thymus, cervical lymph nodes, and long bones of the legs (femur and tibia) were collected at defined and/or humane endpoints, in accordance with protocol. For intracranial tumor-bearing animals, humane endpoints include inability to ambulate two steps forward with prompting. For subcutaneous tumor-bearing animals, humane endpoints include tumor size greater than 20 mm in one dimension, 2000 mm³ in total volume, or tumor ulceration or necrosis. Spleens and thymuses were weighed prior to processing. Briefly, tissues were processed in RPMI, minced into single cell suspensions, cell-strained, counted, stained with antibodies, and analyzed via flow cytometry. Bone marrow cells were flushed out from one femur and one tibia. Blood samples were directly labeled with antibodies and red blood cells subsequently lysed using eBioscience RBC lysis buffer (eBioscience, San Diego, Calif.) or BD Pharm Lyse (BD Biosciences). Spleen and bone marrow were subjected to RBC lysis prior to antibody-labeling, while lymph nodes and thymus were labeled once single cell suspensions were created.

### S1P1 Flow Cytometry

For S1P1 staining, all cell suspensions were prepared in staining buffer with the fixative agent (0.1% Buffered Neutral Formalin (BNF) (Sigma-Aldrich), 0.5% Bovine Serum Albumin (Sigma-Aldrich), and 2 mM EDTA (Gibco) in PBS). Cells were passed through 40 μm nylon mesh cell strainers. After removing RBCs by BD Pharm Lyse lysing solution (BD Biosciences), cells were re-suspended at a density of 5×10⁶ to 2×10⁷ cells per mL in the same staining buffer as described above and were aliquoted in a volume of 100 Cells were then incubated with either rat anti-mouse S1P1 APC-conjugated antibody (R&D systems) or mouse anti-human S1P1 eFlour 660-conjugated antibody (eBioscience) for one hour at 4° C. and were washed once. Next, samples were incubated for 30 minutes at 4° C. with relevant antibody cocktails consisting of antibodies to additional markers (see Reagents). Cells were analyzed with an LSRFortessa (BD Biosciences) and data were analyzed with FlowJo software (Ashland, Oreg.).

### Adoptive Cell Transfer

For tracking cells in vivo, the spleens from naïve C57BL/6 mice were processed into single-cell suspensions in RPMI 1640 (Gibco) containing 10% fetal bovine serum (Gemini Bio-Products). Bone marrow single-cell suspensions from intracranial CT-2A tumor-bearing C57BL/6 mice were acquired from two femurs, two tibias, two humeri, and sternum to achieve maximum yield. Bone marrow cells were then enriched for T-cells via the AutoMACS Pro Separator using the Pan T-Cell Isolation Kit II, mouse with DEPLETE program (Miltenyi Biotec, Auburn, Calif.). Cells from spleens and bone marrow were labeled with CellTrace CFSE (Life Technologies). The labeled cells were transferred IV via tail veins (1×10⁷ cells in 200 μL of PBS per mouse) into tumor-free or intracranial CT-2A tumor bearing C57BL/6 day 18 after tumor implantation. The numbers of CFSE-positive T-cells in the bone marrow were assessed by flow cytometry at specified time points following transfer.

### ELISA

Relevant mice underwent retro-orbital bleed at pre-determined time-points using heparin-coated capillary tubes (VWR). Heparinized blood was then centrifuged and aliquots of plasma were stored at −80° c. S1P1 levels in murine plasma were analyzed using a S1P1 competitive ELISA kit (Echelon Biosciences, Salt Lake City, Utah) according to the manufacturer's instructions.

### Liquid Chromatography-Tandem Mass Spectrometry (LC-MS/MS)

Bone marrow was harvested by removing mouse tibia and femurs, removing the ends of the long bones to expose the marrow cavity, placing the long bones inside a centrifuge tube with a hole in the tip and then nesting it inside another centrifuge tube, and spinning for 10,000 g for 15 seconds to produce a pellet. Sample was then frozen at −80° c. Brains were harvested, frozen with liquid nitrogen, and homogenized using mortar and pestle. Plasma was also collected in EDTA-coated tubes. All samples were delivered to Duke Proteomics and Metabolomics Shared Resource and were analyzed by LC-MS/MS.

### Statistical Analysis

For human studies, the sample size of 15 patients and 15 controls was chosen so that a two-tailed t-test comparing groups has 80% power to detect a difference that is 1.1 times the standard deviation of the outcome variable in each group. For animal studies sample sizes were chosen based on historical experience and were variable based on numbers of surviving mice available at experimental time-points or technical limitations. Female mice aged 6-12 weeks were included in studies, without additional exclusion criteria employed. Mice were pooled and then sequentially assigned to each pertinent group. Animal studies were not blinded. For statistical comparisons, two-tailed paired and unpaired t-tests were generally used to compare groups. When underlying assumptions for these statistical tests were violated, nonparametric alternatives, such as the Wilcoxon signed rank or Wilcoxon rank sum test, were used. Analysis of variance with interaction, χ2 tests, and correlational analyses were also conducted. Bar graphs and dot plots are used to graphically display data, with dot plots used preferentially when group sizes are smaller or data demonstrate non-Gaussian distributions. Bar graphs and dot plots display the mean+/−the standard error of the mean. Survival comparisons were made by Gehan-Breslow-Wilcoxon test. The specific statistical method employed for each data presentation is denoted in the respective figure legends.

### Example 7

### Discussion of Results Described in Examples 1-5

The foregoing examples demonstrate sequestration as a novel mode of T-cell dysfunction in cancer, specifically intracranial tumors. The S1P-S1P1 axis is proposed as the contributing mediator, with S1P1 loss on naïve T-cells fostering their sequestration in bone marrow. Disturbances to T-cell S1P1 are not previously reported in cancer, and T-cell sequestration remains a mostly unaddressed mode of T-cell dysfunction. Sequestration of T-cells may instigate their resultant antigenic ignorance, limiting their anti-tumor capacities.

S1P1 and S1P4 are highly expressed by T-cells, with S1P1 regulating T-cell chemotactic responses, but also impacting resident memory commitment, Treg-induction, and IL-6-dependent pathways. The present data suggest that tumors of the intracranial compartment may usurp a previously unrecognized CNS capacity for eliciting this same phenomenon. Such a capacity may play a physiologic role limiting T-cell access to the CNS and contribute to immune privilege. Interventions targeting S1P1 internalization more specifically may be effective at guiding increased numbers of functional T-cells into intracranial tumors.

Both the lack of observed differences in S1P1 transcript levels in T-cells from tumor-bearing mice, and the improved S1P1 levels seen with hindered receptor internalization, indicate that the defect may be at the protein level, with the disturbance being either increased receptor internalization or delayed/failed receptor recycling. Blockade of known transcriptional down-regulators of S1P1 that are prevalent in GBM, such as TGF-β, produced no effect on sequestration in our hands (data not shown).

S1P1 loss and sequestration characterized predominantly naïve T-cells in our studies. S1P1 stabilization licensed 41BB agonism and PD-1 blockade, the latter of which has already failed in clinical trials for recurrent GBM as a monotherapy. The synergy observed demonstrates that reduced T-cell numbers may be a limiting factor for immunotherapeutic efficacy against intracranial tumors, and that reversal of T-cell sequestration may be useful, including as a therapeutic adjunct. The persistent benefits seen when genetic S1P1 stabilization was replaced with G-CSF imply T-cell sequestration may be averted via available pharmacologic strategies for averting T-cell sequestration.

The present findings indicate that T-cell sequestration may be a contributing factor to T-cell lymphopenia in patients with GBM. While radiation, temozolomide, and dexamethasone may exacerbate T-cell lymphopenia, T-cell disappearances occur earlier and more severely than previously thought, extending to thymus and SLO.

Lastly, the foregoing results indicate that a variety of tumors placed intracranially elicit bone marrow T-cell sequestration, while the same tumors placed peripherally do not exhibit the same proclivity (FIG. 4A). It can be appreciated that the present findings impact immunotherapeutic design not only for GBM patients, but for patients with intracranial metastases as well.

### Example 8

### Genetic Knockouts: β-Arrestin 1 (β-Arr1/BARR1) and β-Arrestin 1 (β-Arr2/BARR2) Inhibition to Prevent S1P1 Internalization

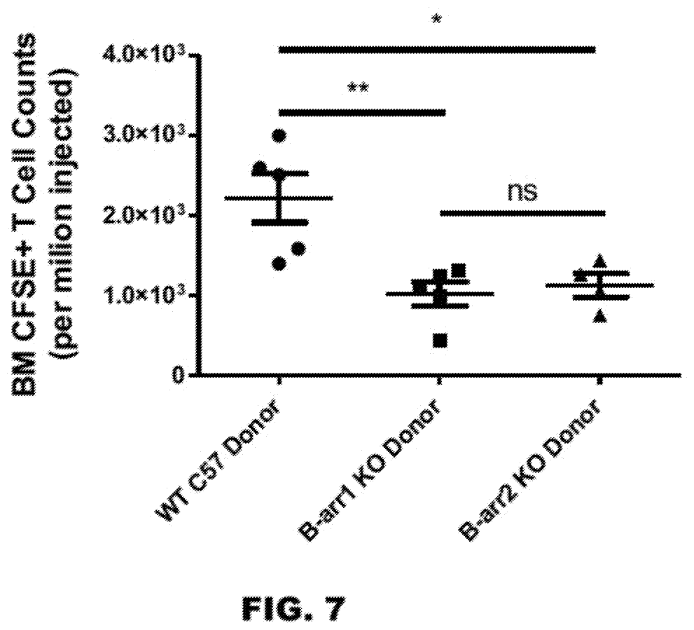

Adoptive transfer of T-cells from BARR1 and BARR2 knockout donors Techniques similar to those described in Example 6 were used to track the trafficking of T-cells with genetic knockout of either BARR1 or BARR2. T-cells from spleens of either BARR1 or BARR2 knockout donors were labeled with CellTrace CFSE (Life Technologies) and injected intravenously via tail veins (1×10⁷ cells in 200 μL of PBS per mouse) into intracranial CT-2A-tumor-bearing wild type C57BL/6 day 18 after tumor implantation. The number of CFSE+ donor T-cells in the bone marrow of recipients was assessed by flow cytometry 24 hours later. When compared to T-cells from wild type C57BL/6 donors, T-cells from both BARR1 and BARR2 knockout donors failed to accumulate in the bone marrow of recipients with intracranial tumors (FIG. 7). This suggests that BARR1 and BARR2 knockout T-cells resist sequestration in bone marrow of CT2A glioma-bearing recipients.

### Example 9

### BARR1 and BARR2 Knockout Mice Bearing CT2A Glioma

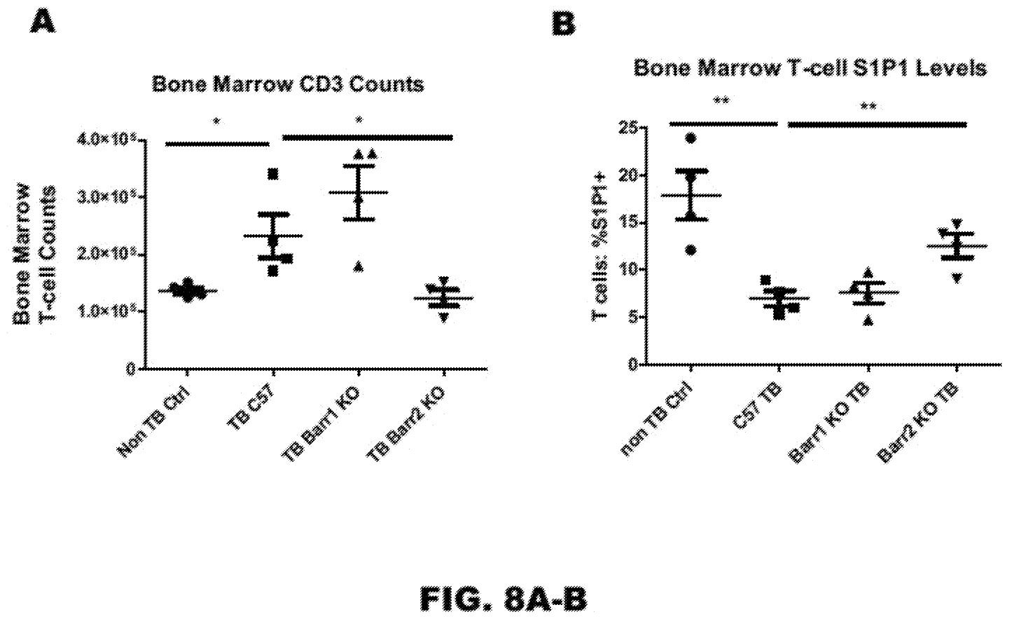

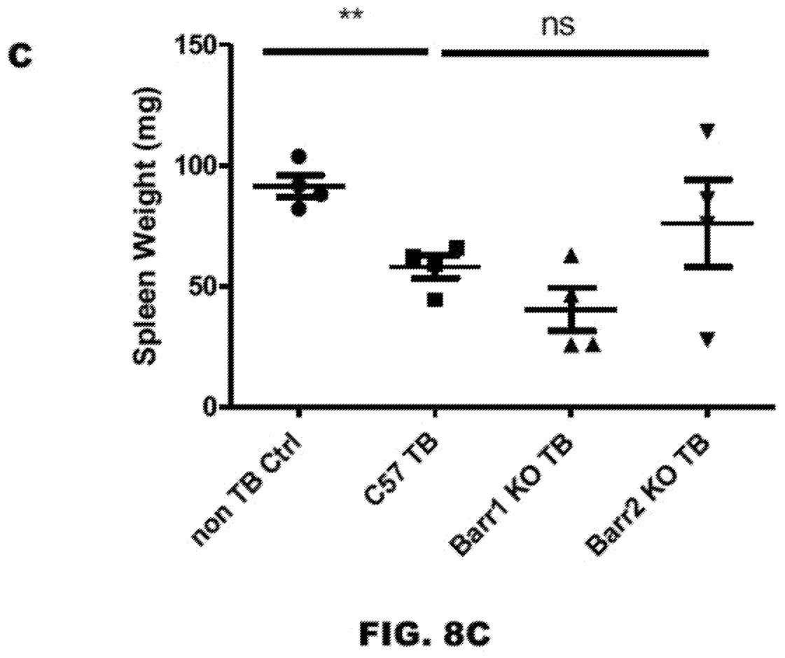

CT-2A murine glioma cells (1×10⁴ in 5 μL) were implanted intracranially into right cerebral hemisphere of BARR1 and BARR2 knockout mice. The number of T-cells in the bone marrow of tumor-bearing mice was determined by flow cytometry on day 18 following tumor implantation. Bone marrow T-cell sequestration, the robust phenotype previously characterized in intracranial CT-2A-bearing wild type C57BL/6, is abrogated in BARR2 knockout mice bearing CT2A, but not in BARR1 knockout mice (FIG. 8A). Also exclusive to BARR2 knockout mice bearing CT2A was a restoration of T-cell S1P1 levels (FIG. 8B) and of spleen volumes (FIG. 8C) to nearly control levels.

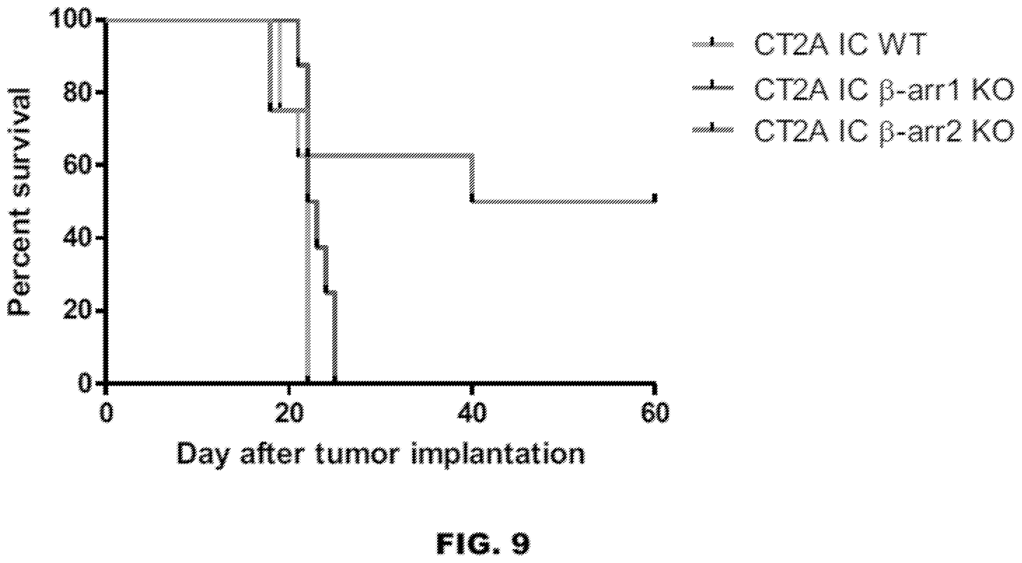

BARR2 knockout mice with CT-2A murine glioma also demonstrated prolonged survival with approximately 50% long-term survivors. These survival benefits were not observed in BARR1 knockout mice (FIG. 9). Of note, for the experiments described in Example 9, BARR1 and BARR2 were knocked-out globally, not just in the T-cells as in the experiments described in Example 8. Reconciling the results from both experimental models suggests counterproductive pleiotropic effects of systemic BARR1 antagonism, while the benefit of BARR2 antagonism is preserved even when inhibition is at a systemic (global) level.

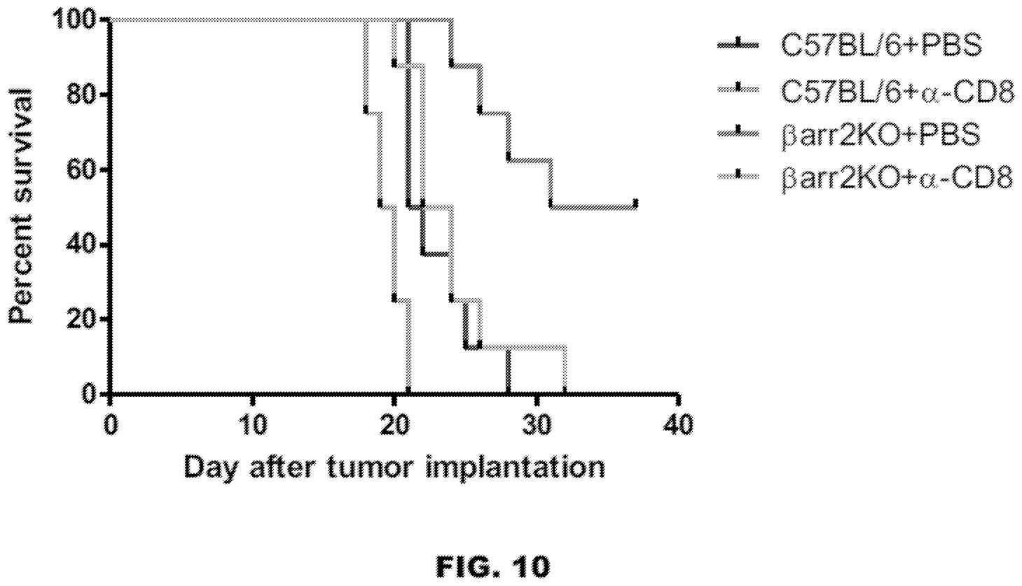

The survival benefit of BARR2 antagonism was abrogated by CD8+ T-cell depletion with anti-CD8+ antibody (Bio-X-cell) treatments (FIG. 10). This result suggests that BARR2 antagonism conveys survival benefits against intracranial tumors in a T-cell dependent manner.

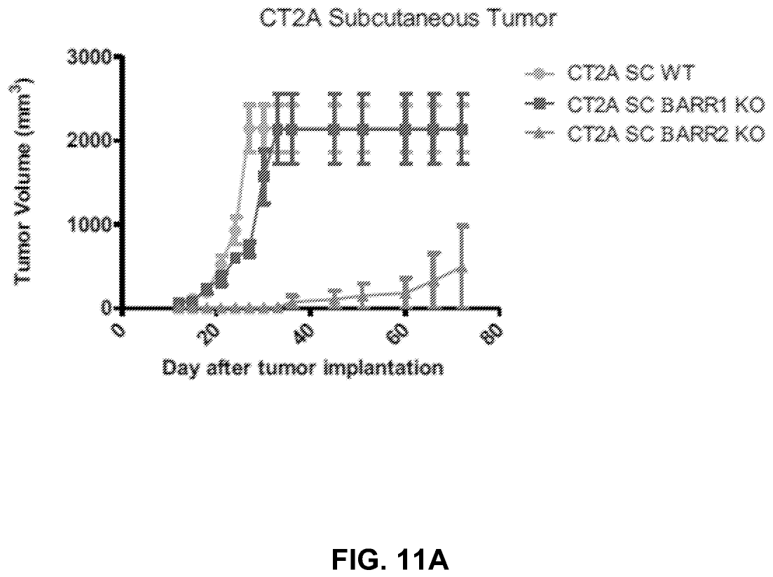

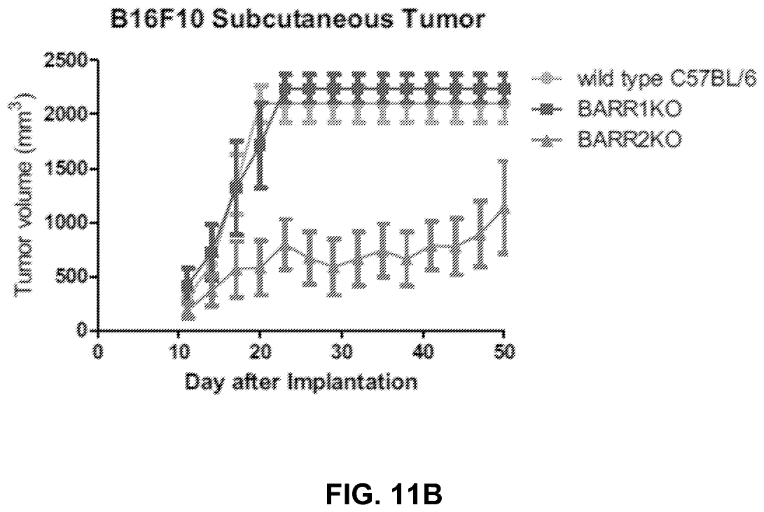

β-arrestin 2 knockout mice, but not β-arrestin 1 knockout mice, show restricted tumor growth in a subcutaneous murine CT2A glioma model, despite absence of T-cell sequestration in the context of subcutaneous tumors, indicating multiple benefits to β-arrestin 2 inhibition beyond just reversal of sequestration (FIG. 11).

### Example 10

Screening of a Small Molecule Library to Identify β-Arrestin Inhibitors that Reverse S1P1 Internalization

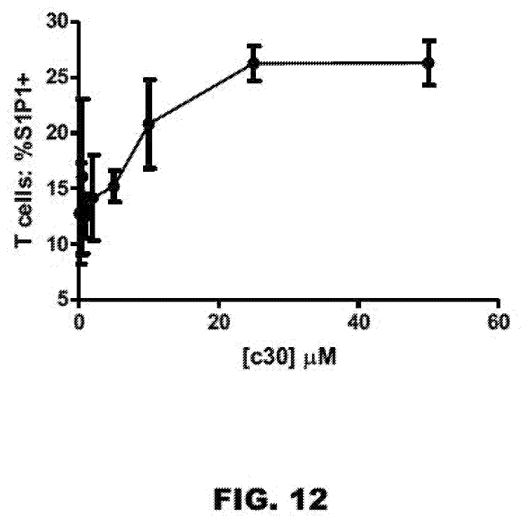

More than 3,500 compounds from a structurally diverse, drug-like compound (DDLC) library containing the National Cancer Institute (NCI) diversity set, natural products, and NCI FDA-approved drugs were initially screened for avid BARR binding using a Fluorescence-based thermal shift assay. The compounds that shift the melting temperatures of the receptor-BARR complex more than 2° c. (both increase and decrease) are considered binders. An initially screened compound (C30) demonstrated the ability to inhibit BARR and increase S1P1 levels on T cells (FIG. 12).

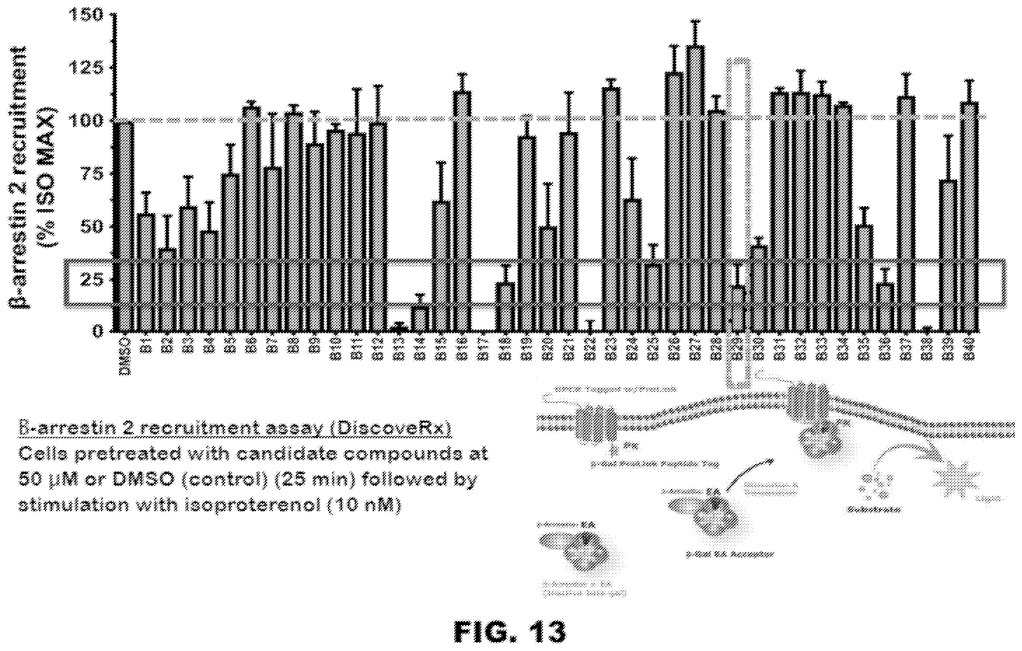

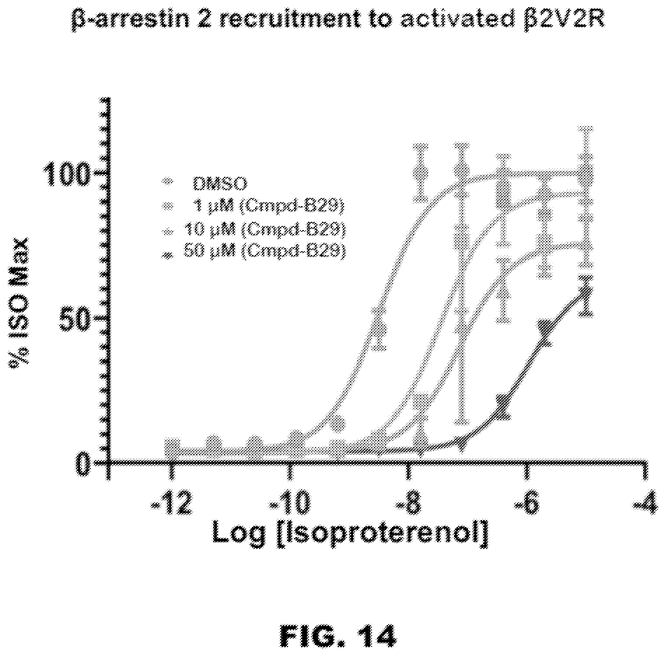

Given the pleiotropic/counterproductive effects of BARR1 antagonism mentioned in Example 9, the next phase of screening was designed to select compounds specific for BARR2 binding. As there is a 70% structural similarity between BARR1 and BARR2, BARR2 binders that were also BARR1 binders were eliminated. Only potential BARR2-specific binders proceeded. Potential BARR2 binders were then also tested/selected for their ability to inhibit BARR2 recruitment using the DiscoveRx assay. DiscoveRx cells expressing chimeric β2-adrenergic receptor (β2AR) with C-terminal tail from vasopressin receptor 2 (β2V2R) that is known to bind β-arrestin 2 very tightly were employed in this assay. The DiscoveRx cells were pretreated with candidate compounds at 50 μM or DMSO (control) for 25 minutes followed by stimulation with isoproterenol (10 nM). A promising compound (B29) inhibits more than 75% of β-arrestin 2 activity induced by isoproterenol, which is a receptor agonist (FIG. 13). Testing B29 further, the DiscoveRx cells were pretreated with compound B29 at 1, 10, 50 μM or DMSO (control) for 25 minutes followed by stimulation with isoproterenol at various concentrations. The titration curves with β-arrestin 2 recruitment activity reveal that B29 shifts the potency of agonist rightward and decreases maximal response in a dose dependent fashion, indicating that it inhibits the β-arrestin 2-induced functional response (FIG. 14).

### Example 11

### In Vivo Testing of β-Arrestin Inhibitors

BARR2 small molecule inhibitor candidates from the in vitro screening above (i.e. B29) are tested for toxicity and efficacy in vivo. Phamacokinetics studies are initially conducted in the CT2A murine model of established glioma. Data are used to initiate Investigational New Drug (IND)-enabling studies with leading BARR2 small molecule inhibitors by themselves, as well as combinatorial strategies with T-cell activating immunotherapies.

The disclosure of every patent, patent application, and publication cited herein is hereby incorporated herein by reference in its entirety for the information indicated in context herein. In the event of a conflict between the disclosure herein and the incorporated matter, the information bodily included in this application is controlling.

### Example 12

### Small Molecules Inhibitors of β-Arrestin 2

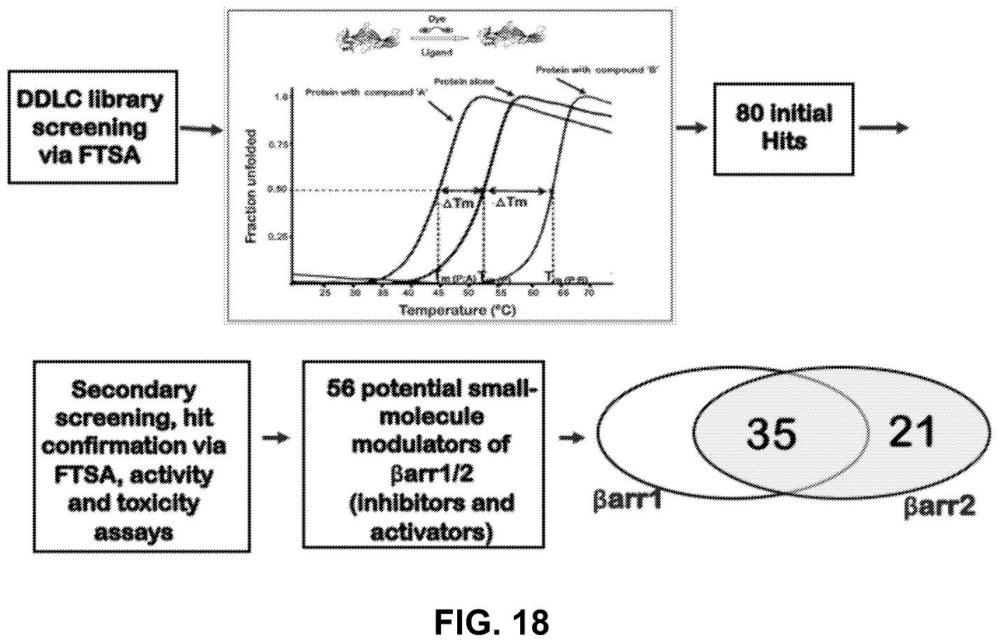

A schematic representation of the process for identifying β-arrestin2 binding small molecule modulators is shown in FIG. 18. The primary screen identified 80 hits that altered the thermal conformational stability of βarr1 or βarr2 by 2° C. compared to controls. Based on secondary confirmation binding, activity and toxicity assays, the 80 initial hits were reduced to 56 hits to undergo further characterization. Thirty-five common binders to both isoforms while 21 bind preferentially to βarr2 under such binding condition.

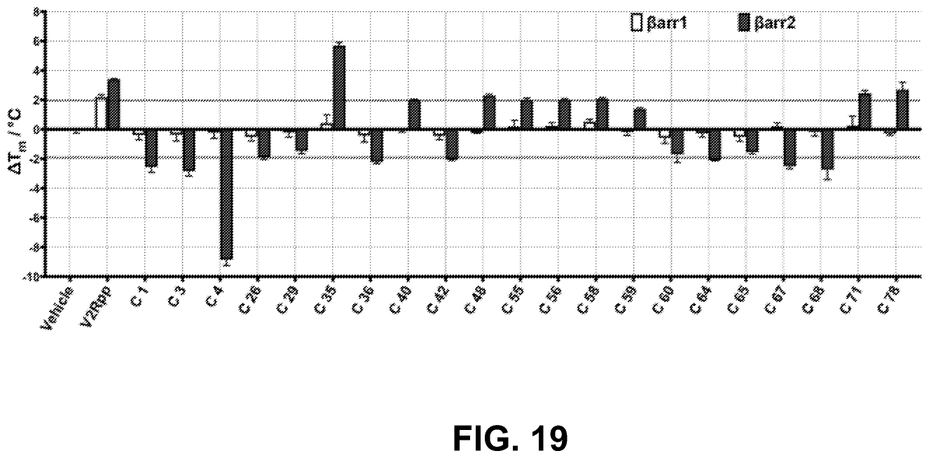

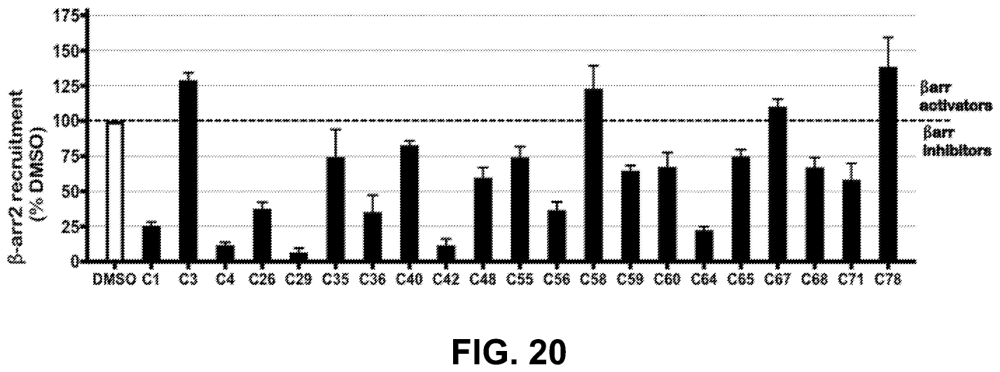

FIG. 19 shows FTSA based binding of 21 small molecule hits to β-arrestin-1 or β-arrestin-2. Plots of the change in melting thermal shift (ΔTm) of βarrs (βarr1 open bar graphs, βarr2 closed bar graphs) in presence of hit compounds (total 21 small-molecules that have preference to bind to barr2 over barr1 under this experimental setting). V2Rpp is a control; βarr1/2 binding phosphorylated peptide which corresponds to the C-terminus of the GPCR, vasopressin-2 receptor (V2R). Compounds scoring ΔTm values approximately ≥2 or ≤−2° C. were considered potential binders to βarr1/2 (dashed lines). All 21 bound preferentially to βarr2 isoform over βarr1. In FIG. 20, the effect of putative βarrs binding compounds (21 hits) on βarrs recruitment to agonist activated GPCR is shown. DiscoveRx-U2OS cells exogenously expressing βarr2 and β2V2R were treated with each putative βarrestin binding compound at 50 μM for 30 min and then stimulated with agonist isoproterenol (10 nM) to induce recruitment of βarr. Data are presented as means±SEM (n=5). The dashed line indicates control agonist alone induced response (10 nM). Above this line indicates compounds that enhance βarr2 activities (activators) and below which compound that inhibit βarrs. Seventeen compounds inhibit isoproterenol-induced βarr2 recruitment to receptor. The remaining 4 either enhance βarr2 activities (C3, C58, and C78) or have little to no effect (C67). Also, as show in FIG. 20, βarr2-inhibiting small molecules inhibited βarr2 recruitment to GPCR activated with isoproterenol.

The influence of inhibitors on the binding of the radio-labeled agonist ³H-Fen to phosphorylated β2V2R (pβ2V2R) was also evaluated using purified proteins constituted in native membranes. Agonist binding to the orthosteric pocket of the receptor increases the receptor binding affinity for transducers (i.e., G proteins and β-arrestins). Subsequent binding of transducers stabilizes the high-affinity state between the receptor and agonist. Thus, radio-ligand binding can be used as a readout for formation of the β-arrestin-receptor complex. We measured radio-ligand binding and found that the addition of βarr2 enhanced the high-affinity agonist (³H-Fen) binding state of the pβ2V2R.

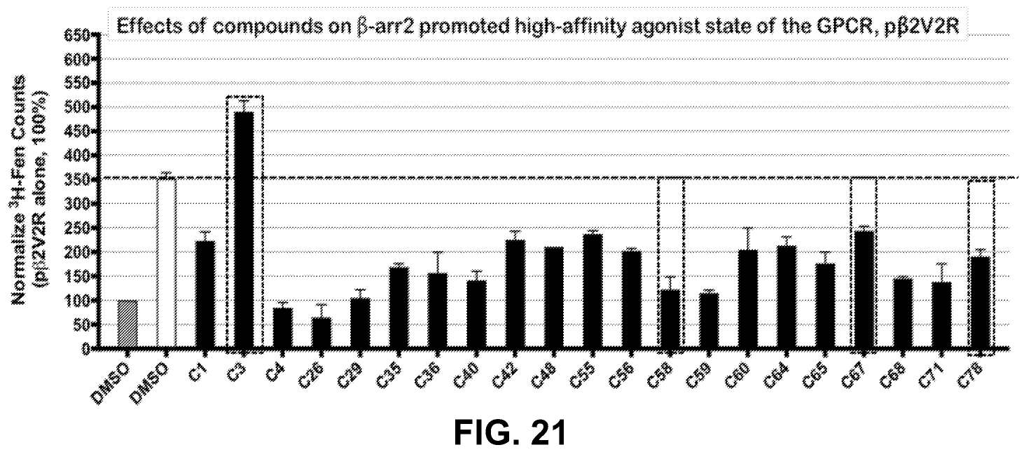

FIG. 21 shows the effects of compounds on βarr2 promoted high-affinity agonist state of the GPCR, pβ2V2R. All 21 compounds were evaluated for their influence on βarr2-promoted high-affinity receptor state in radio-labeled agonist (3H-Fen) binding studies in vitro, using phosphorylated GPCR, β2V2R in membranes. Binding of an agonist at the orthosteric pocket of GPCRs has been previously shown to promote enhanced binding affinity of the βarrs as well as the bound agonist for the receptor. Here, the exogenously added βarr2 enhanced the high-affinity agonist (³H-Fen) binding state of the pβ2V2R (second bar graph/open bar graph). Inhibitor decrease while activator (C3) increase this βarr-promoted high-affinity ³H-Fen binding signals (bar-graphs in black). The first bar graph in each panel is DMSO alone without βarr2. Dashed lines indicate control lines, above which indicates compound that activate and below which compound that inhibit βarr2. Boxed compounds were the ones didn't have inhibitory effects on βarr2-recruitment. All 17 inhibited βarr2-promoted high affinity agonist state of the receptor.

βarrs were recognized to orchestrate a number of intracellular signaling paradigms that occur independent of G protein participation. βarrs are known to mediate ERK1/2 activation by serving as receptor agonist-regulated scaffolds for several signaling components, including the cRaf1-MEK1/2-ERK1/2 MAP kinase cascade. Accordingly, the consequences of pharmacologic inhibition of βarr2 (by all 17 compounds) recruitment to GPCRs on βarr-dependent ERK activation downstream of GPCRs were investigated.

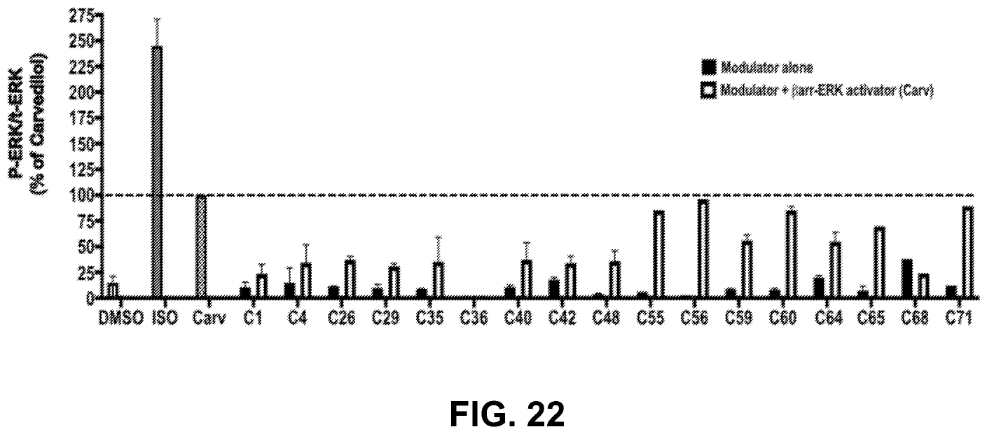

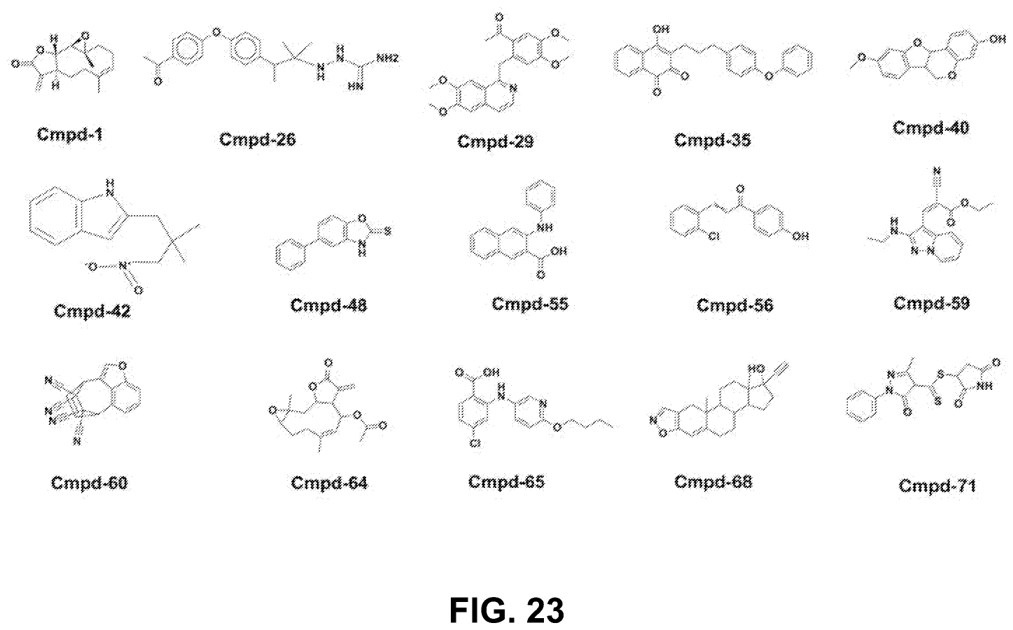

FIG. 22 shows the effects of 21 βarr2-binders on βarr-dependent GPCR mediated ERK MAP kinase activation. The effect of 17 βarr2 binders on βarr-dependent, carvedilol-induced β2-adrenergic receptor (β2AR) mediated ERK phosphorylation in HEK293 cells stably expressing FLAG-tagged β2Ars is shown. Bar graphs showing quantification of ERK activation in presence of vehicle DMSO, 1 μM agonist isoproterenol (ISO), 10 μM of a βarr biased ligand Carvedilol (Cary), 30 μM the compounds alone or together with Carvedilol (Cary). HEK293 cells stably expressing FLAG-tagged β2ARs were pretreated with vehicle or compounds for 30, then stimulated with indicated concentration of carvedilol for 5 min, quenched and analyzed by Western blotting. Data represent the mean±SEM for n independent experiments. DMSO no stimulation; Cary carvedilol; Iso isoproterenol; p-ERK phosphorylated ERK; t-ERK total ERK. Thirteen out of these 17 compounds inhibited Barr-dependent ERK activation while 4 have little to no effects. One compound among these was found to bind to receptor as well (C4) and C36 has cytotoxicity issues (it is an FDA approved drug). Removing C4 and C36 from this list, 15 compounds represent candidates for therapeutic applications.

### Example 13

### βARR2 Depletion Provides Anti-Tumor Efficacy in the Setting of GBM and Other Cancer Models.

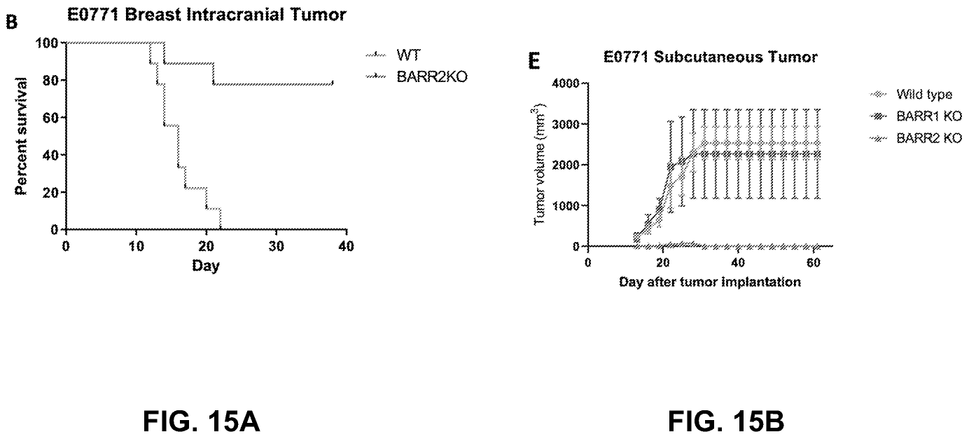

It has been demonstrated that S1P1-stabilized mice with established intracranial tumors have increased number of T-cells at the tumor site. Stabilizing S1P1 on the T-cell surface can synergize and license the anti-tumor capacities of T-cells newly freed from bone marrow when the strategy is coupled to T-cell-activating therapies such as 4-1BB agonism and anti-PD1 (Chongsathidkiet P, et al., Sequestration of T cells in bone marrow in the setting of glioblastoma and other intracranial tumors. Nat Med. 2018; 24(9):1459-68. Epub 2018/08/13. doi: 10.1038/s41591-018-0135-2. PubMed PMID: 30104766; PMCID: PMC6129206). Surprisingly, βARR2 knockout mice demonstrated 30-50% long-term survival to intracranial CT2A glioma in the absence of additional therapies. These survival benefits were not observed in βARR1 knockout mice (FIG. 9). Additionally, markedly extended survival was seen in our model of triple negative breast cancer brain metastasis (IC E0771), with up to 80% long-term survivors in the βARR2 knockout cohort (FIG. 15A). Interestingly, βARR2 knockout mice also showed slower tumor growth in a subcutaneous CT2A murine glioma model (FIG. 11A). Likewise, we saw anti-tumor effects in subcutaneous models of melanoma (B16F10) (FIG. 11B) and triple negative breast cancer (E0771) (FIG. 15B). Therefore, βARR2-deficiency exhibits a survival benefit in both intracranial and subcutaneous tumor models. These data suggest that the mechanism of anti-tumor efficacy extends beyond reversal of bone marrow T-cell sequestration, given that this phenomenon is specific to intracranial tumors and not observed in subcutaneous models.

### βARR2 Deficiency Requires T-Cells to Convey Survival Benefit

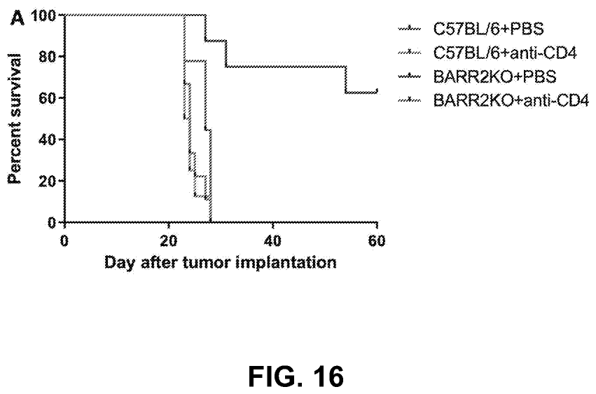

When we depleted T-cells were depleted with anti-CD4 antibodies (FIG. 16) or anti-CD8 antibodies (FIG. 10), the previously seen survival benefit of PARR 2 antagonism in the CT2A model was abrogated. This result suggests, but does not prove, that T-cells mediate the anti-tumor efficacy of βARR2 inhibition. To more stringently investigate this, mice with T cell-specific βARR2 deficiency can be used. These mice will allow better identification of the impact of βARR2 inhibition within T cells. Simultaneously, a bone marrow chimera can be employed to replace the hematopoietic cells of wild-type recipients with those from βARR2 knockout donors. This will serve to investigate the impact of βARR2 inhibition in the hematopoietic compartment more broadly.

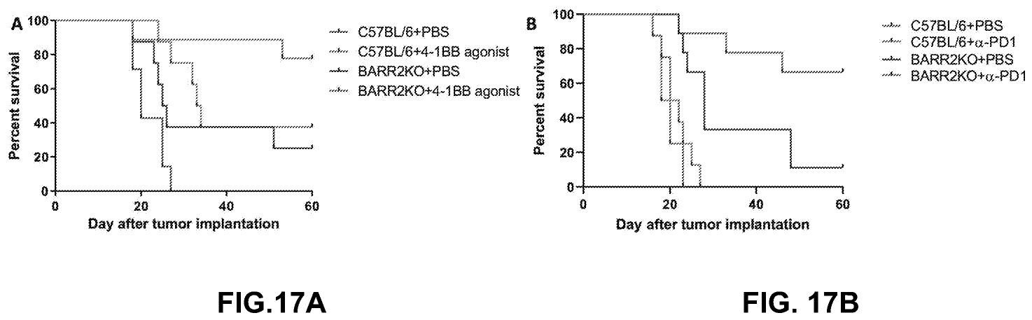

βARR2 depletion synergizes with 4-1BB agonism and checkpoint blockade. To investigate the additive benefits of βARR2 depletion when combined with T-cell activating or checkpoint blockade therapies, we treated intracranial CT2A-bearing βARR2 knockout mice with 4-1BB agonist or PD-1 antagonist antibodies, respectively. βARR2-deficiency synergizes with both 4-1BB agonism (FIG. 17A) and PD-1 antagonism (FIG. 17B) to mediate enhanced efficacy against GBM.

### Example 14

### Compounds for Pharmaceutical Compositions and Methods of the Invention

Table 2, below, shows compound designations used herein, along with their corresponding IUPAC names and PubChem CIDs.

TABLE 3
Chemical Names

| CMPD | | PubChem C |
|---|---|---|
| ID | IUPAC Name | ID: |
| C1 | (7Z)-4,8-dimethyl-12-methylidene-3,14- | 5353864 |
| | dioxatricyclo[9.3.0.02,4]tetradec-7-en-13-one | |
| C26 | 2-[(E)-1-[4-(4- | 5351213 |
| | acetylphenoxy)phenyl]ethylideneamino]guani- | |
| | dine; nitrate | |
| C29 | 1-[2-[(6,7-dimethoxyisoquinolin-1-yl)methyl]- | 72351 |
| | 4,5-dimethoxyphenyl]ethanone | |
| C35 | 4-hydroxy-3-[3-(4-phenoxyphenyl)propyl]naph- | 219294 |
| | thalene-1,2-dione | |
| C40 | (6aR,11aR)-9-methoxy-6a,11a-dihydro-6H- | 350085 |
| | [1]benzofuro[3,2-c]chromen-3-ol | |
| C42 | 2-[(E)-2-nitroethenyl]-1H-indole | 5382764 |
| C48 | 5-phenyl-3H-1,3-benzoxazole-2-thione | 3032663 |
| C55 | 3-anilinonaphthalene-2-carboxylic acid | 138888 |
| C56 | 3-(2-chlorophenyl)-1-(4-hydroxyphenyl)prop-2- | 224570 |
| | en-1-one | |
| C59 | ethyl (Z)-2-cyano-3-[2-(ethylamino)pyrazolo[1,5- | 36688489 |
| | a]pyridin-3-yl]prop-2-enoate | |
| C60 | 4-oxatetracyclo[8.2.2.12,5.09,15]pentadeca- | 363912 |
| | 2,5,7,9(15),13-pentaene-11,11,12,12-tetracar- | |
| | bonitrile | |
| C64 | [(8Z)-3,8-dimethyl-12-methylidene-13-oxo-4,14- | 5860420 |
| | dioxatricyclo[9.3.0.03,5]tetradec-8-en-10-yl] | |
| | acetate | |
| C65 | 2-[(6-butoxypyridin-3-yl)amino]-4-chlorobenzoic | 246853 |
| | acid | |
| C68 | 17-ethynyl-2,18-dimethyl-7-oxa-6- | 2949 |
| | azapentacyclo[11.7.0.02,10.04,8.014,18]icosa- | |
| | 4(8),5,9-trien-17-ol | |
| C71 | (2,5-dioxopyrrolidin-3-yl) 3-methyl-5-oxo-1- | 327585 |
| | phenyl-4H-pyrazole-4-carbodithioate | |

Compound structures for the compounds shown in Table 3 are provided in FIG. 20.

Compound 30 as disclosed herein can be useful according to the methods of the invention, as a β-arrestin inhibitor. Compound 30 comprises, consists of, or consists essentially of the general formula (I) (termed Cmpd 30; ((Z)-3-((furan-2-ylmethyl)imino)-N,N-dimethyl-3H-1,2,4-dithiazol-5-amine)):

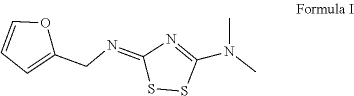

or a pharmaceutically acceptable salt, solvate, hydrate, prodrug, or derivative thereof.

Compound B29 as disclosed herein can be useful according to the methods of the invention, as a β-arrestin inhibitor showing selectivity for BARR2. Compound B29 comprises, consists of, or consists essentially of the general formula (II) (termed Cmpd B29; (1-(2-((6,7-dimethoxyisoquinolin-1-yl)methyl)-4,5-dimethoxyphenyl)ethan-1-one)):

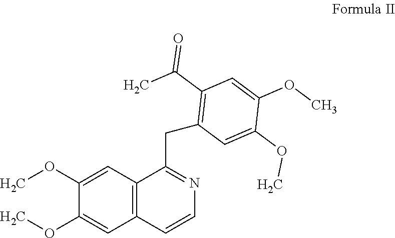

or a pharmaceutically acceptable salt, solvate, hydrate, prodrug, or derivative thereof.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method for treating an intracranial disease, the method comprising enhancing egress of T-cells from bone marrow of a subject in need thereof.

2. The method of claim 1, wherein the T-cells comprise surface displayed sphingosine-1-phosphate receptor 1 (S1P1), and wherein the method comprises increasing the interactions between S1P1 and sphingosine-1-phosphate (S1P).

3. The method of claim 1 or 2, wherein the method comprises promoting S1P1 display on the surface of the T-cells.

4. The method of any one of claims 1-3, wherein the method comprises stabilizing S1P1 on the surface of the T-cells.

5. The method of any one of claims 1-4, wherein the method comprises reducing internalization of S1P1 from the surface of the T-cells.

6. The method of any one of claims 1-5, wherein the T-cells are naïve T-cells.

7. The method of any one of claims 1-6, wherein the T-cells are CD4 and/or CD8 T-cells.

8. The method of any one of claims 1-7, wherein the method comprises inhibiting an interaction between S1P1 and β-arrestin.

9. The method of any one of claims 1-8, wherein the method comprises administering a β-arrestin inhibitor to the subject.

10. The method of claim 9, wherein the β-arrestin inhibitor comprises a β-arrestin 1 inhibitor or a β-arrestin 2 inhibitor.

11. The method of any one of claims 1-10, wherein the method comprises inhibiting GRK2-mediated phosphorylation of S1P1.

12. The method of any one of claims 1-11, wherein the method comprises inhibiting clathrin-mediated endocytosis of S1P1.

13. The method of any one of claims 1-12, further comprising administering a 41BB agonist and/or a PD-1 blockade to the subject.

14. The method of any one of claims 1-13, further comprising administering a granulocyte colony-stimulating factor to the subject.

15. The method of any one of claims 1-14, wherein the subject is a human.

16. The method of any one of claims 1-15, wherein the intracranial disease is a primary intracranial tumor, an intracranial metastatic tumor, an inflammatory brain disease or disorder, a stroke, or a traumatic brain injury.

17. The method of any one of claims 1-16, wherein the intracranial disease is glioblastoma.

18. A pharmaceutical composition comprising an agent that promotes surface display of sphingosine-1-phosphate receptor 1 (S1P1) on a T-cell.

19. The pharmaceutical composition of claim 18, wherein the agent increases the interaction between S1P1 and sphingosine-1-phosphate (S1P).

20. The pharmaceutical composition of claim 18 or 19, wherein the agent stabilizes S1P1 on the surface of the T-cell.

21. The pharmaceutical composition of any one of claims 18-20, wherein the agent reduces internalization of S1P1 from the surface of the T-cell.

22. The pharmaceutical composition of any one of claims 18-21, wherein the agent inhibits an interaction between S1P1 and arrestin.

23. The pharmaceutical composition of any one of claims 18-22, wherein the agent comprises a β-arrestin inhibitor.

24. The pharmaceutical composition of any one of claims 18-23, wherein the agent comprises a β-arrestin 1 inhibitor or a β-arrestin 2 inhibitor.

25. The pharmaceutical composition of any one of claims 18-22, wherein the agent inhibits GRK2-mediated phosphorylation of S1P1.

26. The pharmaceutical composition of any one of claims 18-22, wherein the agent inhibits clathrin-mediated endocytosis of S1P1.

27. The pharmaceutical composition of claim 24, wherein the agent is (Z)-3-((furan-2-ylmethyl)imino)-N,N-dimethyl-3H-1,2,4-dithiazol-5-amine) (compound C30) of general formula I:

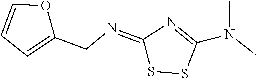

28. The pharmaceutical composition of claim 24, wherein the agent is a β-arrestin 2 inhibitor.

29. The pharmaceutical composition of claim 28, wherein the agent is 1-(2-((6,7-dimethoxyisoquinolin-1-yl)methyl)-4,5-dimethoxyphenyl)ethan-1-one) (compound B29) of general formula II:

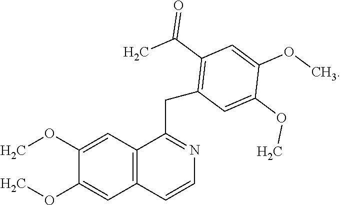

30. A method of treating a disease or a disorder associated with T-cell sequestration in the bone marrow in a subject in need thereof, the method comprising administering a pharmaceutical composition comprising a β-arrestin inhibitor in an amount effective to release the T-cells from sequestration.

31. A method of treating a disease or a disorder associated with loss of sphingosine-1-phosphate receptor 1 (S1P1) expression on the surface of T-cells in a subject in need thereof, the method comprising administering a β-arrestin inhibitor in an amount effective to stabilize S1P1 levels on the T-cells by hindering S1P1 internalization.

32. A method for mobilizing T-cells sequestered in the bone marrow into circulation in a subject in need thereof, the method comprising administering a β-arrestin inhibitor in an amount effective to release the T-cells into circulation.

33. A method for reversing T-cell ignorance in a subject in need thereof, the method comprising administering a β-arrestin inhibitor in an amount effective to stabilize S1P1 levels on the T-cells, thereby reversing the ignorance.

34. A method for treating cancer in a subject in need thereof, comprising administering a β-arrestin inhibitor.

35. The method of claim 34, wherein the β-arrestin inhibitor inhibits β-arrestin 2.

36. The method of claim 34, wherein the β-arrestin inhibitor inhibits β-arrestin 2 but not β-arrestin 1.

37. The method of any one of claims 34-36, wherein the β-arrestin inhibitor is selected from the group consisting of compounds C 1, C26, C29, C35, C40, C42, C48, C55, C56, C59, C60, C64, C65, C68, C71, and combinations thereof.

38. The method of any one of claims 34-37, wherein the β-arrestin inhibitor is C29.

39. A method of diagnosis of intracranial tumors, the method comprising determining the presence of S1P1 on the surface of T cells, wherein a loss of surface S1P1 on the T cells indicates the presence of or advancement of the intracranial tumor.
